SEQUENCE LISTING (90 sequences)

SEQ ID NO: 1 - protein, 116 aa, Artificial Sequence
  Synthetic Sequence
  MISC_FEATURE (4)..(4): Xaa1 is leucine (Leu) or phenylalanine (Phe)
  MISC_FEATURE (5)..(5): Xaa2 is valine (Val), methionine (Met), or leucine (Leu)
  MISC_FEATURE (44)..(44): Xaa3 is arginine (Arg) or glycine (Gly)
  MISC_FEATURE (56)..(56): Xaa4 is glycine (Gly), serine (Ser), or alanine (Ala)
  MISC_FEATURE (72)..(72): Xaa5 is arginine (Arg) or alanine (Ala)
  MISC_FEATURE (74)..(74): Xaa6 is threonine (Thr) or lysine (Lys)
  MISC_FEATURE (84)..(84): Xaa7 is serine (Ser) or alanine (Ala)
  MISC_FEATURE (95)..(95): Xaa8 is tyrosine (Tyr) or phenylalanine (Phe)
QVQXXQSGAEVKKPGASVKVSCKASGFTFTSYDINWVRQAPGQXLEWMGWIYPGDXSTKYNEKFKGRVTITXDXSASTAYMELXSLRSEDTAVYXCTRSFYTMDYWGQGTTVTVSS

SEQ ID NO: 2 - protein, 116 aa, Artificial Sequence
  Synthetic Sequence
QVQLVQSGAEVKKPGASVKVSCKASGFTFTSYDINWVRQAPGQRLEWMGWIYPGDGSTKYNEKFKGRVTITRDTSASTAYMELSSLRSEDTAVYYCTRSFYTMDYWGQGTTVTVSS

SEQ ID NO: 3 - protein, 116 aa, Artificial Sequence
  Synthetic Sequence
QVQLVQSGAEVKKPGASVKVSCKASGFTFTSYDINWVRQAPGQGLEWMGWIYPGDGSTKYNEKFKGRVTITADKSASTAYMELSSLRSEDTAVYFCTRSFYTMDYWGQGTTVTVSS

SEQ ID NO: 4 - protein, 116 aa, Artificial Sequence
  Synthetic Sequence
QVQLMQSGAEVKKPGASVKVSCKASGFTFTSYDINWVRQAPGQRLEWMGWIYPGDSSTKYNEKFKGRVTITRDKSANTAYMELASLRSEDTAVYFCTRSFYTMDYWGQGTTVTVSS

SEQ ID NO: 5 - protein, 116 aa, Artificial Sequence
  Synthetic Sequence
QVQLMQSGAEVKKPGASVKVSCKASGFTFTSYDINWVRQAPGQRLEWMGWIYPGDASTKYNEKFKGRVTITRDKSANTAYMELASLRSEDTAVYFCTRSFYTMDYWGQGTTVTVSS

SEQ ID NO: 6 - protein, 116 aa, Artificial Sequence
  Synthetic Sequence
QVQLMQSGAEVKKPGASVKVSCKASGFTFTSYDINWVRQAPGQRLEWMGWIYPGDSSTKYNEKFKGRVTITADKSANTAYMELASLRSEDTAVYFCTRSFYTMDYWGQGTTVTVSS

SEQ ID NO: 7 - protein, 116 aa, Artificial Sequence
  Synthetic Sequence
QVQLLQSGAEVKKPGASVKVSCKASGFTFTSYDINWVRQAPGQRLEWMGWIYPGDSSTKYNEKFKGRVTITRDKSANTAYMELSSLRSEDTAVYFCTRSFYTMDYWGQGTTVTVSS

SEQ ID NO: 8 - protein, 116 aa, Artificial Sequence
  Synthetic Sequence
QVQLLQSGAEVKKPGASVKVSCKASGFTFTSYDINWVRQAPGQRLEWMGWIYPGDSSTKYNEKFKGRVTITADKSANTAYMELASLRSEDTAVYFCTRSFYTMDYWGQGTTVTVSS

SEQ ID NO: 9 - protein, 116 aa, Artificial Sequence
  Synthetic Sequence
QVQLLQSGAEVKKPGASVKVSCKASGFTFTSYDINWVRQAPGQRLEWMGWIYPGDASTKYNEKFKGRVTITRDKSANTAYMELASLRSEDTAVYFCTRSFYTMDYWGQGTTVTVSS

SEQ ID NO: 10 - protein, 116 aa, Artificial Sequence
  Synthetic Sequence
QVQLLQSGAEVKKPGASVKVSCKASGFTFTSYDINWVRQAPGQRLEWMGWIYPGDSSTKYNEKFKGRVTITRDKSANTAYMELASLRSEDTAVYFCTRSFYTMDYWGQGTTVTVSS

SEQ ID NO: 11 - protein, 116 aa, Artificial Sequence
  Synthetic Sequence
QVQFLQSGAEVKKPGASVKVSCKASGFTFTSYDINWVRQAPGQRLEWMGWIYPGDSSTKYNEKFKGRVTITADKSANTAYMELSSLRSEDTAVYFCTRSFYTMDYWGQGTTVTVSS

SEQ ID NO: 12 - protein, 116 aa, Artificial Sequence
  Synthetic Sequence
QVQFVQSGAEVKKPGASVKVSCKASGFTFTSYDINWVRQAPGQRLEWMGWIYPGDSSTKYNEKFKGRVTITRDKSANTAYMELSSLRSEDTAVYFCTRSFYTMDYWGQGTTVTVSS

SEQ ID NO: 13 - protein, 116 aa, Artificial Sequence
  Synthetic Sequence
QVQFVQSGAEVKKPGASVKVSCKASGFTFTSYDINWVRQAPGQRLEWMGWIYPGDSSTKYNEKFKGRVTITADKSANTAYMELSSLRSEDTAVYFCTRSFYTMDYWGQGTTVTVSS

SEQ ID NO: 14 - protein, 116 aa, Artificial Sequence
  Synthetic Sequence
QVQLLQSGAEVKKPGASVKVSCKASGFTFTSYDINWVRQAPGQRLEWMGWIYPGDSSTKYNEKFKGRVTITRDKSANTAYMELSSLRSEDTAVYYCTRSFYTMDYWGQGTTVTVSS

SEQ ID NO: 15 - protein, 120 aa, Artificial Sequence
  Synthetic Sequence
  MISC_FEATURE (33)..(33): Xaa1 is tryptophan (Trp) or tyrosine (Tyr)
  MISC_FEATURE (35)..(35): Xaa2 is histidine (His), asparagine (Asn), or tyrosine (Tyr)
  MISC_FEATURE (42)..(42): Xaa3 is glycine (Gly) or arginine (Arg)
  MISC_FEATURE (52)..(52): Xaa4 is aspartic acid (Asp), glutamic acid (Glu), or histidine (His)
  MISC_FEATURE (54)..(54): Xaa5 is serine (Ser), threonine (Thr), or tyrosine (Tyr)
  MISC_FEATURE (55)..(55): Xaa6 is asparagine (Asn) or glycine (Gly)
  MISC_FEATURE (56)..(56): Xaa7 is serine (Ser), alanine (Ala), or aspartic acid (Asp)
QVQLVQSGAEVKKPGASVKVSCKASGYTFTNYXMXWVRQAPXQGLEWMGMFXPXXXVTRLNQKFKDRVTMTRDTSTSTVYMELSSLRSEDTAVYYCARTTSMIIGGFAYWGQGTLVTVSS

SEQ ID NO: 16 - protein, 120 aa, Artificial Sequence
  Synthetic Sequence
QVQLVQSGAEVKKPGASVKVSCKASGYTFTNYWMHWVRQAPGQGLEWMGMFDPSNSVTRLNQKFKDRVTMTRDTSTSTVYMELSSLRSEDTAVYYCARTTSMIIGGFAYWGQGTLVTVSS

SEQ ID NO: 17 - protein, 120 aa, Artificial Sequence
  Synthetic Sequence
QVQLVQSGAEVKKPGASVKVSCKASGYTFTNYWMHWVRQAPGQGLEWMGMFEPSNAVTRLNQKFKDRVTMTRDTSTSTVYMELSSLRSEDTAVYYCARTTSMIIGGFAYWGQGTLVTVSS

SEQ ID NO: 18 - protein, 120 aa, Artificial Sequence
  Synthetic Sequence
QVQLVQSGAEVKKPGASVKVSCKASGYTFTNYWMNWVRQAPRQGLEWMGMFEPSNAVTRLNQKFKDRVTMTRDTSTSTVYMELSSLRSEDTAVYYCARTTSMIIGGFAYWGQGTLVTVSS

SEQ ID NO: 19 - protein, 120 aa, Artificial Sequence
  Synthetic Sequence
QVQLVQSGAEVKKPGASVKVSCKASGYTFTNYWMYWVRQAPRQGLEWMGMFHPSNAVTRLNQKFKDRVTMTRDTSTSTVYMELSSLRSEDTAVYYCARTTSMIIGGFAYWGQGTLVTVSS

SEQ ID NO: 20 - protein, 120 aa, Artificial Sequence
  Synthetic Sequence
QVQLVQSGAEVKKPGASVKVSCKASGYTFTNYWMNWVRQAPRQGLEWMGMFHPSNAVTRLNQKFKDRVTMTRDTSTSTVYMELSSLRSEDTAVYYCARTTSMIIGGFAYWGQGTLVTVSS

SEQ ID NO: 21 - protein, 120 aa, Artificial Sequence
  Synthetic Sequence
QVQLVQSGAEVKKPGASVKVSCKASGYTFTNYYMNWVRQAPRQGLEWMGMFHPTGDVTRLNQKFKDRVTMTRDTSTSTVYMELSSLRSEDTAVYYCARTTSMIIGGFAYWGQGTLVTVSS

SEQ ID NO: 22 - protein, 120 aa, Artificial Sequence
  Synthetic Sequence
QVQLVQSGAEVKKPGASVKVSCKASGYTFTNYWMNWVRQAPRQGLEWMGMFHPTGDVTRLNQKFKDRVTMTRDTSTSTVYMELSSLRSEDTAVYYCARTTSMIIGGFAYWGQGTLVTVSS

SEQ ID NO: 23 - protein, 120 aa, Artificial Sequence
  Synthetic Sequence
QVQLVQSGAEVKKPGASVKVSCKASGYTFTNYYMNWVRQAPRQGLEWMGMFHPYGDVTRLNQKFKDRVTMTRDTSTSTVYMELSSLRSEDTAVYYCARTTSMIIGGFAYWGQGTLVTVSS

SEQ ID NO: 24 - protein, 120 aa, Artificial Sequence
  Synthetic Sequence
QVQLVQSGAEVKKPGASVKVSCKASGYTFTNYWMNWVRQAPRQGLEWMGMFHPYGDVTRLNQKFKDRVTMTRDTSTSTVYMELSSLRSEDTAVYYCARTTSMIIGGFAYWGQGTLVTVSS

SEQ ID NO: 25 - protein, 117 aa, Artificial Sequence
  Synthetic Sequence
  MISC_FEATURE (1)..(1): Xaa1 is glutamine (Gln) or aspartic acid (Asp)
  MISC_FEATURE (4)..(4): Xaa2 is leucine (Leu) or phenylalanine (Phe)
  MISC_FEATURE (25)..(25): Xaa3 is threonine (Thr) or serine (Ser)
  MISC_FEATURE (41)..(41): Xaa4 is proline (Pro) or phenylalanine (Phe)
  MISC_FEATURE (44)..(44): Xaa5 is lysine (Lys) or asparagine (Asn)
  MISC_FEATURE (45)..(45): Xaa6 is glycine (Gly) or lysine (Lys)
  MISC_FEATURE (71)..(71): Xaa7 is serine (Ser) or threonine (Thr)
  MISC_FEATURE (72)..(72): Xaa8 is valine (Val) or arginine (Arg)
  MISC_FEATURE (95)..(95): Xaa9 is tyrosine (Tyr) or phenylalanine (Phe)
XVQXQESGPGLVKPSQTLSLTCTVXGYSITSDFAWNWIRQXPGXXLEWIGYISYSGDTNYNPSLKSRVTIXXDTSKNQFSLKLSSVTAADTAVYXCAIRGPYSFTYWGQGTLVTVSS

SEQ ID NO: 26 - protein, 117 aa, Artificial Sequence
  Synthetic Sequence
QVQLQESGPGLVKPSQTLSLTCTVSGYSITSDFAWNWIRQPPGKGLEWIGYISYSGDTNYNPSLKSRVTISVDTSKNQFSLKLSSVTAADTAVYYCAIRGPYSFTYWGQGTLVTVSS

SEQ ID NO: 27 - protein, 117 aa, Artificial Sequence
  Synthetic Sequence
QVQFQESGPGLVKPSQTLSLTCTVSGYSITSDFAWNWIRQPPGKGLEWIGYISYSGDTNYNPSLKSRVTISRDTSKNQFSLKLSSVTAADTAVYFCAIRGPYSFTYWGQGTLVTVSS

SEQ ID NO: 28 - protein, 117 aa, Artificial Sequence
  Synthetic Sequence
QVQLQESGPGLVKPSQTLSLTCTVSGYSITSDFAWNWIRQFPGNKLEWIGYISYSGDTNYNPSLKSRVTISVDTSKNQFSLKLSSVTAADTAVYYCAIRGPYSFTYWGQGTLVTVSS

SEQ ID NO: 29 - protein, 117 aa, Artificial Sequence
  Synthetic Sequence
QVQFQESGPGLVKPSQTLSLTCTVSGYSITSDFAWNWIRQPPGNKLEWIGYISYSGDTNYNPSLKSRVTISRDTSKNQFSLKLSSVTAADTAVYFCAIRGPYSFTYWGQGTLVTVSS

SEQ ID NO: 30 - protein, 117 aa, Artificial Sequence
  Synthetic Sequence
QVQFQESGPGLVKPSQTLSLTCTVSGYSITSDFAWNWIRQFPGKGLEWIGYISYSGDTNYNPSLKSRVTISRDTSKNQFSLKLSSVTAADTAVYFCAIRGPYSFTYWGQGTLVTVSS

SEQ ID NO: 31 - protein, 117 aa, Artificial Sequence
  Synthetic Sequence
QVQFQESGPGLVKPSQTLSLTCTVSGYSITSDFAWNWIRQFPGNKLEWIGYISYSGDTNYNPSLKSRVTISRDTSKNQFSLKLSSVTAADTAVYFCAIRGPYSFTYWGQGTLVTVSS

SEQ ID NO: 32 - protein, 117 aa, Artificial Sequence
  Synthetic Sequence
DVQFQESGPGLVKPSQTLSLTCTVTGYSITSDFAWNWIRQPPGKGLEWIGYISYSGDTNYNPSLKSRVTITRDTSKNQFSLKLSSVTAADTATYFCAIRGPYSFTYWGQGTLVTVSS

SEQ ID NO: 33 - protein, 116 aa, Artificial Sequence
  Synthetic Sequence
QVQLQQSGPELVKPGALVKISCKASGFTFTSYDINWVRQRPGQGLEWIGWIYPGDGSTKYNEKFKGKATLTADKSSSTAYMQLSSLTSENSAVYFCTRSFYTMDYWGQGTSVTVSS

SEQ ID NO: 34 - protein, 120 aa, Artificial Sequence
  Synthetic Sequence
QVQLQQSGPELVRPGASVRMSCKASDYTFTNYWMHWVKQRPGQGLEWIGMFDPSNSVTRLNQKFKDKATLNVDKSSNTAYIQFSSLTFEDSAVYYCARTTSMIIGGFAYWGQGTLVTVSS

SEQ ID NO: 35 - protein, 117 aa, Artificial Sequence
  Synthetic Sequence
DVQFQESGPGLVKPSQSLSLTCTVTGYSITSDFAWNWIRQFPGNKLEWMGYISYSGDTNYNPSLKSRISITRDTSKNQFFLQLNSVTTEDTATYFCAIRGPYSFTYWGPGTLVTVSS

SEQ ID NO: 36 - protein, 112 aa, Artificial Sequence
  Synthetic Sequence
  MISC_FEATURE (34)..(34): Xaa1 is glycine (Gly) or alanine (Ala)
  MISC_FEATURE (41)..(41): Xaa2 is phenylalanine (Phe) or tyrosine (Tyr)
  MISC_FEATURE (54)..(54): Xaa3 is tyrosine (Tyr) or serine (Ser)
DIVMTQSPLSLPVTPGEPASISCRSSKSLLHSNXNTYLYWXLQKPGQSPQLLIXRMSNLASGVPDRFSGSGSGTDFTLKISRVEAEDVGVYYCMQHLEYPFTFGQGTKLEIK

SEQ ID NO: 37 - protein, 112 aa, Artificial Sequence
  Synthetic Sequence
DIVMTQSPLSLPVTPGEPASISCRSSKSLLHSNGNTYLYWYLQKPGQSPQLLIYRMSNLASGVPDRFSGSGSGTDFTLKISRVEAEDVGVYYCMQHLEYPFTFGQGTKLEIK

SEQ ID NO: 38 - protein, 112 aa, Artificial Sequence
  Synthetic Sequence
DIVMTQSPLSLPVTPGEPASISCRSSKSLLHSNGNTYLYWFLQKPGQSPQLLISRMSNLASGVPDRFSGSGSGTDFTLKISRVEAEDVGVYYCMQHLEYPFTFGQGTKLEIK

SEQ ID NO: 39 - protein, 112 aa, Artificial Sequence
  Synthetic Sequence
DIVMTQSPLSLPVTPGEPASISCRSSKSLLHSNANTYLYWFLQKPGQSPQLLISRMSNLASGVPDRFSGSGSGTDFTLKISRVEAEDVGVYYCMQHLEYPFTFGQGTKLEIK

SEQ ID NO: 40 - protein, 112 aa, Artificial Sequence
  Synthetic Sequence
  MISC_FEATURE (32)..(32): Xaa1 is serine (Ser) or arginine (Arg)
  MISC_FEATURE (34)..(34): Xaa2 is glycine (Gly) or alanine (Ala)
  MISC_FEATURE (43)..(43): Xaa3 is glutamine (Gln) or histidine (His)
DIVMTQTPLSLSVTPGQPASISCRSSKSLLHXNXITYFYWYLXKPGQPPQLLIYQMSNLASGVPDRFSGSGSGTDFTLKISRVEAEDVGVYYCAQNLELPLTFGGGTKVEIK

SEQ ID NO: 41 - protein, 112 aa, Artificial Sequence
  Synthetic Sequence
DIVMTQTPLSLSVTPGQPASISCRSSKSLLHSNGITYFYWYLQKPGQPPQLLIYQMSNLASGVPDRFSGSGSGTDFTLKISRVEAEDVGVYYCAQNLELPLTFGGGTKVEIK

SEQ ID NO: 42 - protein, 112 aa, Artificial Sequence
  Synthetic Sequence
DIVMTQTPLSLSVTPGQPASISCRSSKSLLHSNAITYFYWYLQKPGQPPQLLIYQMSNLASGVPDRFSGSGSGTDFTLKISRVEAEDVGVYYCAQNLELPLTFGGGTKVEIK

SEQ ID NO: 43 - protein, 112 aa, Artificial Sequence
  Synthetic Sequence
DIVMTQTPLSLSVTPGQPASISCRSSKSLLHRNAITYFYWYLQKPGQPPQLLIYQMSNLASGVPDRFSGSGSGTDFTLKISRVEAEDVGVYYCAQNLELPLTFGGGTKVEIK

SEQ ID NO: 44 - protein, 112 aa, Artificial Sequence
  Synthetic Sequence
DIVMTQTPLSLSVTPGQPASISCRSSKSLLHRNAITYFYWYLHKPGQPPQLLIYQMSNLASGVPDRFSGSGSGTDFTLKISRVEAEDVGVYYCAQNLELPLTFGGGTKVEIK

SEQ ID NO: 45 - protein, 107 aa, Artificial Sequence
  Synthetic Sequence
  MISC_FEATURE (64)..(64): Xaa1 is serine (Ser) or proline (Pro)
  MISC_FEATURE (71)..(71): Xaa2 is phenylalanine (Phe) or tyrosine (Tyr)
DIQMTQSPSSLSASVGDRVTITCRASQDINNYLNWYQQKPGKAPKLLIYYTSRLHSGVPSRFSXSGSGTDXTFTISSLQPEDIATYYCQQGHTLPWTFGGGTKVEIK

SEQ ID NO: 46 - protein, 107 aa, Artificial Sequence
  Synthetic Sequence
DIQMTQSPSSLSASVGDRVTITCRASQDINNYLNWYQQKPGKAPKLLIYYTSRLHSGVPSRFSGSGSGTDFTFTISSLQPEDIATYYCQQGHTLPWTFGGGTKVEIK

SEQ ID NO: 47 - protein, 107 aa, Artificial Sequence
  Synthetic Sequence
DIQMTQSPSSLSASVGDRVTITCRASQDINNYLNWYQQKPGKAPKLLIYYTSRLHSGVPSRFSPSGSGTDYTFTISSLQPEDIATYYCQQGHTLPWTFGGGTKVEIK

SEQ ID NO: 48 - protein, 112 aa, Artificial Sequence
  Synthetic Sequence
DIVMTQAAPSVPVTPGESVSISCRSSKSLLHSNGNTYLYWFLQRPGQSPQLLISRMSNLASGVPDRFSGSGSGTAFTLRISRVEAEDVGVYYCMQHLEYPFTFGSGTKLEIK

SEQ ID NO: 49 - protein, 112 aa, Artificial Sequence
  Synthetic Sequence
DIVMTQAAFSNPVTLGTSASISCRSSKSLLHSNGITYFYWYLQRPGQSPQLLIYQMSNLASGVPDRFSGSGSGTDFTLRISRVEAEDVGIYYCAQNLELPLTFGAGTKLELK

SEQ ID NO: 50 - protein, 107 aa, Artificial Sequence
  Synthetic Sequence
DIQMTQTTSSLSASLGDRVTINCRASQDINNYLNWYQQKPDGTIRLLIYYTSRLHSGVPSRFSPNGSGTDYSLTINNLEQEDIATYFCQQGHTLPWTFGGGTKLEIK

SEQ ID NO: 51 - protein, 117 aa, Artificial Sequence
  Synthetic Sequence
DVQFQESGPGLVKPSQTLSLTCTVTGYSITSDFAWNWIRQPPGKGLEWIGYISYSGDTNYNPSLKSRVTITRDTSKNQFSLKLSSVTAADTAVYFCAIRGPYSFTYWGQGTLVTVSS

SEQ ID NO: 52 - protein, 118 aa, Artificial Sequence
  Synthetic Sequence
DVQFQESGPGLVKPSQTLSLTCTVTGYSITADFAWNWIRQPPGKGLEWIGYISYSGDTNYNPSLKSRVTITRDTSKNQFSLKLSSVTAADTAVYFCAIRGPYSFTYWGQGTLVTVSSA

SEQ ID NO: 53 - protein, 118 aa, Artificial Sequence
  Synthetic Sequence
DLQFQESGPGLVKPSQTLSLTCTVTGYSITADFAWNWIRQPPGKGLEWIGYISYSGDTNYNPSLKSRVTITRDTSKNQFSLKLSSVTAADTAVYFCAIRGPYSFTYWGQGTLVTVSSA

SEQ ID NO: 54 - protein, 118 aa, Artificial Sequence
  Synthetic Sequence
DLQFQESGPGLVKPSQTLSLTCTVSRYSITADFAWNWIRQPPGKGLEWIGYISYSGDTNYNPSLKSRVTITRDTSKNQFSLKLSSVTAADTAVYFCAIRGPYSFTYWGQGTLVTVSSA

SEQ ID NO: 55 - protein, 109 aa, Artificial Sequence
  Synthetic Sequence
DIQMTQSPSSLSASVGDRVTITCRASQWINNYLNWYQQKPGKAPKLLIYYTSMLHSGVPSRFSPSGSGNDYTFTISSLQPEDIATYYCQQGHTLPWTFGGGTKVEIKRT

SEQ ID NO: 56 - protein, 116 aa, Artificial Sequence
  Synthetic Sequence
  MISC_FEATURE (4)..(4): Xaa1 is leucine (Leu) or phenylalanine (Phe)
  MISC_FEATURE (5)..(5): Xaa2 is valine (Val), methionine (Met), or leucine (Leu)
  MISC_FEATURE (44)..(44): Xaa3 is arginine (Arg) or glycine (Gly)
  MISC_FEATURE (56)..(56): Xaa4 is glycine (Gly), serine (Ser), or alanine (Ala)
  MISC_FEATURE (72)..(72): Xaa5 is arginine (Arg) or alanine (Ala)
  MISC_FEATURE (74)..(74): Xaa6 is threonine (Thr) or lysine (Lys)
  MISC_FEATURE (77)..(77): Xaa7 is serine (Ser) or asparagine (Asn)
  MISC_FEATURE (84)..(84): Xaa8 is serine (Ser) or alanine (Ala)
  MISC_FEATURE (95)..(95): Xaa9 is tyrosine (Tyr) or phenylalanine (Phe)
QVQXXQSGAEVKKPGASVKVSCKASGFTFTSYDINWVRQAPGQXLEWMGWIYPGDXSTKYNEKFKGRVTITXDXSAXTAYMELXSLRSEDTAVYXCTRSFYTMDYWGQGTTVTVSS

SEQ ID NO: 57 - protein, 118 aa, Artificial Sequence
  Synthetic Sequence
  MISC_FEATURE (1)..(1): Xaa1 is glutamine (Gln) or aspartic acid (Asp)
  MISC_FEATURE (2)..(2): Xaa2 is valine (Val) or leucine (Leu)
  MISC_FEATURE (4)..(4): Xaa3 is leucine (Leu) or phenylalanine (Phe)
  MISC_FEATURE (25)..(25): Xaa4 is threonine (Thr) or serine (Ser)
  MISC_FEATURE (26)..(26): Xaa5 is glycine (Gly) or arginine (Arg)
  MISC_FEATURE (31)..(31): Xaa6 serine (Ser) or alanine (Ala)
  MISC_FEATURE (41)..(41): Xaa7 is proline (Pro) or phenylalanine (Phe)
  MISC_FEATURE (44)..(44): Xaa8 is lysine (Lys) or asparagine (Asn)
  MISC_FEATURE (45)..(45): Xaa9 is glycine (Gly) or lysine (Lys)
  MISC_FEATURE (71)..(71): Xaa10 is serine (Ser) or threonine (Thr)
  MISC_FEATURE (72)..(72): Xaa11 is valine (Val) or arginine (Arg)
  MISC_FEATURE (93)..(93): Xaa12 is threonine (Thr) or valine (Val)
  MISC_FEATURE (95)..(95): Xaa13 is tyrosine (Tyr) or phenylalanine (Phe)
  MISC_FEATURE (118)..(118): Xaa14 is is alanine (Ala) or absent
XXQXQESGPGLVKPSQTLSLTCTVXXYSITXDFAWNWIRQXPGXXLEWIGYISYSGDTNYNPSLKSRVTIXXDTSKNQFSLKLSSVTAADTAXYXCAIRGPYSFTYWGQGTLVTVSSX

SEQ ID NO: 58 - protein, 109 aa, Artificial Sequence
  Synthetic Sequence
  MISC_FEATURE (28)..(28): Xaa1 is aspartic acid (Asp) or tryptophan (Trp)
  MISC_FEATURE (53)..(53): Xaa2 is arginine (Arg) or methionine (Met)
  MISC_FEATURE (64)..(64): Xaa3 is glycine (Gly), serine (Ser) or proline (Pro)
  MISC_FEATURE (69)..(69): Xaa4 is threonine (Thr) or asparagine (Asn)
  MISC_FEATURE (71)..(71): Xaa5 is phenylalanine (Phe) or tyrosine (Tyr)
  MISC_FEATURE (108)..(108): Xaa6 is arginine (Arg) or absent
  MISC_FEATURE (109)..(109): Xaa7 is threonine (Thr) or absent
DIQMTQSPSSLSASVGDRVTITCRASQXINNYLNWYQQKPGKAPKLLIYYTSXLHSGVPSRFSXSGSGXDXTFTISSLQPEDIATYYCQQGHTLPWTFGGGTKVEIKXX

SEQ ID NO: 59 - protein, 9 aa, Artificial Sequence
  synthetic
FTFTSYDIN

SEQ ID NO: 60 - protein, 17 aa, Artificial Sequence
  synthetic
WIYPGDGSTKYNEKFKG

SEQ ID NO: 61 - protein, 17 aa, Artificial Sequence
  synthetic
WIYPGDSSTKYNEKFKG

SEQ ID NO: 62 - protein, 17 aa, Artificial Sequence
  synthetic
WIYPGDASTKYNEKFKG

SEQ ID NO: 63 - protein, 7 aa, Artificial Sequence
  synthetic
SFYTMDY

SEQ ID NO: 64 - protein, 9 aa, Artificial Sequence
  synthetic
YTFTNYWMH

SEQ ID NO: 65 - protein, 9 aa, Artificial Sequence
  synthetic
YTFTNYWMN

SEQ ID NO: 66 - protein, 9 aa, Artificial Sequence
  synthetic
YTFTNYWMY

SEQ ID NO: 67 - protein, 9 aa, Artificial Sequence
  synthetic
YTFTNYYMN

SEQ ID NO: 68 - protein, 17 aa, Artificial Sequence
  synthetic
MFDPSNSVTRLNQKFKD

SEQ ID NO: 69 - protein, 17 aa, Artificial Sequence
  synthetic
MFEPSNAVTRLNQKFKD

SEQ ID NO: 70 - protein, 17 aa, Artificial Sequence
  synthetic
MFHPSNAVTRLNQKFKD

SEQ ID NO: 71 - protein, 17 aa, Artificial Sequence
  synthetic
MFHPTGDVTRLNQKFKD

SEQ ID NO: 72 - protein, 11 aa, Artificial Sequence
  synthetic
TTSMIIGGFAY

SEQ ID NO: 73 - protein, 10 aa, Artificial Sequence
  synthetic
YSITSDFAWN

SEQ ID NO: 74 - protein, 10 aa, Artificial Sequence
  synthetic
YSITADFAWN

SEQ ID NO: 75 - protein, 16 aa, Artificial Sequence
  synthetic
YISYSGDTNYNPSLKS

SEQ ID NO: 76 - protein, 8 aa, Artificial Sequence
  synthetic
RGPYSFTY

SEQ ID NO: 77 - protein, 16 aa, Artificial Sequence
  synthetic
RSSKSLLHSNGNTYLY

SEQ ID NO: 78 - protein, 16 aa, Artificial Sequence
  synthetic
RSSKSLLHSNANTYLY

SEQ ID NO: 79 - protein, 6 aa, Artificial Sequence
  synthetic
RMSNLA

SEQ ID NO: 80 - protein, 9 aa, Artificial Sequence
  synthetic
MQHLEYPFT

SEQ ID NO: 81 - protein, 16 aa, Artificial Sequence
  synthetic
RSSKSLLHSNGITYFY

SEQ ID NO: 82 - protein, 16 aa, Artificial Sequence
  synthetic
RSSKSLLHSNAITYFY

SEQ ID NO: 83 - protein, 16 aa, Artificial Sequence
  synthetic
RSSKSLLHRNAITYFY

SEQ ID NO: 84 - protein, 7 aa, Artificial Sequence
  synthetic
QMSNLAS

SEQ ID NO: 85 - protein, 9 aa, Artificial Sequence
  synthetic
AQNLELPLT

SEQ ID NO: 86 - protein, 11 aa, Artificial Sequence
  synthetic
RASQDINNYLN

SEQ ID NO: 87 - protein, 11 aa, Artificial Sequence
  synthetic
RASQWINNYLN

SEQ ID NO: 88 - protein, 7 aa, Artificial Sequence
  synthetic
YTSRLHS

SEQ ID NO: 89 - protein, 7 aa, Artificial Sequence
  synthetic
YTSMLHS

SEQ ID NO: 90 - protein, 9 aa, Artificial Sequence
  synthetic
QQGHTLPWT

(19) United States
(12) Patent Application Publication
(10) Pub. No.: US 2024/0084021 A1
(43) Pub. Date: Mar. 14, 2024

(54) ANTI-INTERLEUKIN 36 RECEPTOR (IL-36R) THERAPY FOR HIDRADENITIS SUPPURATIVA

(71) Applicant: AnaptysBio, Inc., San Diego, CA (US)

(72) Inventor: Paul F. Lizzul, Oak Park, CA (US)

(21) Appl. No.: 18/260,692

(22) PCT Filed: Jan. 7, 2022
(86) PCT No.: PCT/US2022/011695

§ 371 Date: Jul. 7, 2023

Related U.S. Application Data
(60) Provisional application No. 63/135,229, filed on Jan. 8, 2021.

(51) Int. Cl. C07K 16/28

(52) U.S. Cl. CPC C07K 16/2866; C07K 2317/33; C07K 2317/76

(57) ABSTRACT

The invention relates to methods of treating hidradenitis suppurativa in a subject with an inhibitor of the interleukin 36 (IL-36) pathway, and methods of selecting a subject for therapy with the IL-36 pathway inhibitor.

### CROSS-REFERENCE TO RELATED APPLICATIONS

This patent application claims the benefit of U.S. Provisional Patent Application No. 63/135,229, filed Jan. 8, 2021, which is incorporated by reference herein in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 76,909 Byte ASCII (Text) file named “758760_ST25.TXT,” created on Jan. 4, 2022.

### BACKGROUND OF THE INVENTION

Hidradenitis suppurativa is a chronic skin condition characterized by inflamed areas typically located around the armpits and groin. These inflamed areas often include painful lesions, nodules and boils, and usually occur where certain sweat glands (known as apocrine sweat glands) are located. Many patients experience discharge from these lesions. Patients with hidradenitis suppurativa experience a higher risk of cutaneous, extracutaneous and systemic infections. They also have increased rates of depression and greater feelings of stigmatization, resulting in an overall decrease in quality of life. The exact cause of hidradenitis suppurativa is unknown, but follicular occlusion, an inflammatory disorder, (Frew et al., J. Investigative Dermatology (2020)), and genetic factors have all been thought to play a role in development of the disease (Goldburg et al., J. Am. Academy Dermatology 82(5): 1045 (2020)).

There is currently no known cure for hidradenitis suppurativa. Therapy has been limited to biologics, antibiotics, and surgical remedies. Currently adalimumab is the only biologic therapy approved for treatment with patients with hidradenitis suppurativa. However, no treatment has been effective for the vast majority of patients, and many patients have found that none of the current available treatments are effective.

There remains a significant unmet medical need for an effective treatment for hidradenitis suppurativa.

### BRIEF SUMMARY OF THE INVENTION

Provided herein is a method of treating hidradenitis suppurativa in a subject by inhibiting interleukin 36 (IL-36) signaling in the subject, thereby treating the hidradenitis suppurativa. Also provided is a method of selecting a subject with hidradenitis suppurativa for treatment with an inhibitor of the IL-36 pathway on the basis of elevated expression of one or more IL-36 cytokines or IL-36R (or mRNA encoding same), which selection method can optionally be used in conjunction with the method of treating hidradenitis suppurativa in a subject provided herein. The disclosure further provides an IL-36R binding agent and composition comprising same for use to treat hidradenitis suppurativa in the described methods. These and other aspects of the invention will be apparent to the skilled person reading the detailed description.

### DETAILED DESCRIPTION OF THE INVENTION

Provided herein is a method of treating hidradenitis suppurativa in a subject, the method comprising inhibiting IL-36 signaling in the subject, thereby treating the hidradenitis suppurativa. Inhibiting IL-36 signaling can be accomplished by any suitable method that reduces IL-36 receptor (IL-36R) binding to one or more (or all) IL-36 cytokines (e.g., IL-36α, IL-36β, and/or IL-36γ).

The IL-36 cytokines IL-36α, IL-36β, and IL-36γ (formerly IL-1F6, IL-1F8, and IL-1F9) are interleukin-1 (IL-1) family members that bind to the IL-36R (formerly IL-1Rrp2 or IL-1RL2), a receptor of the IL-1R family, and use IL-1 receptor accessory protein (IL-1RAcP) as a coreceptor to stimulate intracellular signals similar to those induced by IL-1 (Towne et al., J. Biol. Chem., 279(14): 13677-13688 (2004)). IL-1F5 is an IL-1 family member that has been shown to act as an antagonist of IL-36R, and is now referred to as IL-36Ra (Dinarello et al., Nat. Immunol., 11(11): 973 (2010)).

The subject can be a mammal, such as a human or non-human primate, with hidradenitis suppurativa. In some embodiments, the hidradenitis suppurativa may be associated with upregulation of IL-36α, IL-36β, and/or IL-36γ compared to a normal subject. Thus, in some embodiments, the subject to be treated can have elevated levels of one or more of IL-36α, IL-36β, and/or IL-36γ compared to a normal, non-diseased subject (e.g., elevated levels of one or more of IL-36α, IL-36β, and/or IL-36γ in a tissue (skin or skin lesion) or fluid sample from an affected tissue of the subject as compared to level of the same cytokine(s) from the same type of sample from a non-affected tissue of the same subject or from a normal, non-diseased subject). In some embodiments, the increased skin expression of the IL-36 cytokine (e.g., IL-36α, IL-36β, and/or IL-36γ) in the sample from affected tissue is at least about 25% higher (e.g., at least about 30% higher, or at least about 50% higher) than the normal baseline expression of the non-affected sample.

In addition, or alternatively, the hidradenitis suppurativa may be associated with upregulation of IL-17 and/or TNF-α. Thus, in some embodiments, the subject to be treated can have elevated levels of one or more of IL-17 and/or TNF-α compared to a normal, non-diseased subject (e.g., elevated levels of one or more of IL-17 and/or TNF-α in a tissue (skin) or fluid sample from an affected tissue of the subject as compared to level of the same cytokine(s) from the same type of sample from a non-affected tissue of the same subject or from a normal, non-diseased subject).

In this respect, and according to a related aspect of the disclosure, there is provided herein a method for selecting a subject (e.g., a mammal or human) with hidradenitis suppurativa for treatment with an inhibitor of the IL-36 pathway. In some embodiments, the method comprises comparing the expression of at least one of an IL-36 cytokine, IL-36R, or mRNA encoding same in a tissue (skin or skin lesion) or fluid sample from an affected tissue of the subject with a suitable normal control (e.g., the level of the same cytokine(s) or receptor or mRNA from the same type of sample from a non-affected tissue of the same subject or from a normal, non-diseased subject, or average of such expression levels in a population of normal, non-diseased subjects). The subject is selected for treatment if the expression of the IL-36 cytokine, IL-36R, or mRNA encoding same is increased (e.g., by at least about 20%, 50%, 100%, 200%, or more) in the subject as compared to the normal control. In other embodiments, the method can comprise comparing the expression of at least one of an IL-36 cytokine, IL-36R, or mRNA encoding same in a tissue (skin or skin lesion) or fluid sample from an affected tissue of the subject before and after administration of an IL-36 pathway inhibitor; and selecting the subject for treatment when a decrease in expression of at least one of an IL-36 cytokine, IL-36R, or mRNA encoding same is observed after administration of the IL-36 pathway inhibitor as compared to before administration of the IL-36 pathway inhibitor. The subject so selected can, optionally, be treated according to the methods described herein.

The IL-36 cytokine and IL-36R protein levels or mRNA levels of the subject (or tissue/fluid sample from the subject) can be measured using any suitable method known in the art. Such methods include, for example, radioimmunoassay (RIA), FACS, or PCR. Normal or standard expression values of the IL-36 cytokine or IL-36R can be established by any suitable technique, e.g., by combining a sample comprising, or suspected of comprising, IL-36 cytokine or IL-36R with an IL-36 cytokine-specific or IL-36R-specific antibody under conditions suitable to form an antigen-antibody complex. The antibody may be directly or indirectly labeled with a detectable substance to facilitate detection of the bound or unbound antibody. Suitable detectable substances include various enzymes, prosthetic groups, fluorescent materials, luminescent materials, and radioactive materials (see, e.g., Zola, Monoclonal Antibodies: A Manual of Techniques, CRC Press, Inc. (1987)).

The methods provided herein can be used to treat Hidradenitis Suppurativa of any degree of severity. In some embodiments, the subject prior to treatment has an abscess and inflammatory nodule (AN) count of at least 1 (e.g., at least 2, at least 3, at least 4, at least 5, at least 6, at least 7, at least 8, at least 9, at least 10, at least 10, at least 11, at least 12, at least 13, at least 14, at least 15, at least 16, at least 17, at least 18, or at least 19 or more). In some embodiments, the subject has an AN count of at least 5 prior to treatment.

As used herein, the terms “treatment,” “treating,” and the like refer to obtaining a desired pharmacologic and/or physiologic effect. For example, the treatment reduces the severity or slows the onet or progression of one or more adverse symptoms of hidradenitis suppurativa by any manner or degree, including, but not limited to, a reduction in the number, frequency, onset, recurrence or severity of lesions, abscesses, nodules, and draining tunnels (also known as sinus tracts or fistulas). Reduction in adverse symptoms can be determined by any suitable technique, including but not limited to a reduction in the AN count of the subject, or by the hidradenitis suppurativa clinical response (HiSCR) (e.g., HiSCR50 or HiSCR75), Sartorius Score or Modified Sartorius Score, Hurley Staging, International Severity Scoring System (IHS4), Dermatology Life Quality Index (DLQI), Hidradenitis Suppurativa-Physician's Global Assessment (HS-PGA), Hidradenitis Suppurativa pain numerical rating score (HS pain NRS), Patient Global Impression of Change (PGIC), or Patient Global Impression of Severity (PGIC).

IL-36 signaling can be inhibited by any suitable method, such as by administering an IL-36 pathway inhibitor that inhibits or neutralizes the IL-36 pathway, e.g., at the level of the cytokines (i.e., inhibits or neutralizes the biological activity of IL-36α, IL-36β, or IL-36γ), the receptor (i.e., inhibits or neutralizes the biological activity of IL-36R), or inhibits or neutralizes the subsequent intracellular signaling induced by the IL-36 cytokines or IL-36R. Any IL-36 pathway inhibitor can be used, provided the inhibitor has an effect that is sufficiently rapid and persistent to allow for a therapeutic effect within the dosing parameters described herein.

The method can include, for instance, administering to a subject in need thereof an agent that specifically binds to IL-36R; an agent that specifically binds to an IL-36 cytokine (e.g., IL-36α, IL-36β, or IL-36γ); or a combination thereof. Examples of IL-36 pathway inhibitors including antibodies or antigen binding fragments thereof that bind to an IL-36 cytokine (e.g., IL-36α, IL-36β, or IL-36γ) or IL-36R, several of which are known in the art including sepsolimab (Boehringer Ingleheim), ANB-019 (AnaptysBio, Inc.), and any of the compounds and compositions disclosed in WO2016168542A1; WO 2020018503 A2; WO2019177883A2; WO2018183173A1; U.S. Ser. No. 10/550,189B2; U.S. Pat. No. 9,023,995B2; or WO2013074569A1, all of which are hereby incorporated by reference.

In an embodiment, the IL-36 pathway inhibitor is an IL-36R binding agent, such as an antibody or antigen-binding antibody fragment, comprising an immunoglobulin heavy chain polypeptide (or at least variable region thereof) and an immunoglobulin light chain polypeptide (or at least variable region thereof). The IL-36R binding agent can be, for example, any binding agent described in WO2016168542A1.

A whole immunoglobulin typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H}1, C_{H}2, and C_{H}3) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical immunoglobulin, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other. As used herein, an immunoglobulin heavy chain or light chain “polypeptide” is used to refer to a whole immunoglobulin polypeptide or a portion or region thereof (e.g., a variable region).

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the hypervariable or complementary determining regions (CDRs). There are four framework regions in each variable domain, which are designated FR1, FR2, FR3, and FR4. The framework regions form the (3 sheets that provide the structural framework of the variable region (see, e.g., C. A. Janeway et al. (eds.), Immunobiology, 5th Ed., Garland Publishing, New York, NY (2001)).

The framework regions are connected by three complementarity determining regions (CDRs). As discussed above, the three CDRs, known as CDR1, CDR2, and CDR3, form the “hypervariable region” of an antibody, which is responsible for antigen binding. The CDR regions also can be referred to using an “H” or “L” in the nomenclature to denote the heavy or light chain, respectively, i.e., CDRH1, CDRH2, CDRH3, CDRL1, CDRL2, or CDRL3. The CDRs of a given Ig sequence can be determined by any of several conventional numbering schemes, such as Kabat, Chothia, Martin (Enhanced Chothia), IGMT, or AHo (see, e.g., Kabat, et al., Sequences of Proteins of Immunological Interest, U.S. Department of Health and Human Services, NIH (1991); Chothia, et al., Canonical Structures for the Hypervariable Regions of Immunoglobulins, J. Mol. Biol., 196:901-917 (1987); Al-Lazikani et al., Standard Conformations for the Canonical Structures of Immunoglobulins, J. Mol. Biol., 273:927-948 (1997); Abhinandan et al., Analysis and Improvements to Kabat and Structurally Correct Numbering of Antibody Variable Domains, Mol. Immunol., 45: 3832-3839 (2008); Lefranc et al., The IMGT unique numbering for immunoglobulins, T cell Receptors and Ig-like domains, The Immunologist, 7: 132-136 (1999); Lefranc et al., IMGT unique numbering for immunoglobulin and T cell receptor variable domains and I superfamily V-like domains, Dev. Comp. Immunol., 27: 55-77 (2003); and Honegger et al., Yet another numbering scheme for immunoglobulin variable domains: an automatic modeling and analysis tool, J. Mol. Biol. 309: 657-670 (2001).

In one embodiment, the immunoglobulin heavy chain variable region comprises, consists of, or consists essentially of the amino acid sequence of Gln Val Gln Xaa1 Xaa2 Gln Ser Gly Ala Glu Val Lys Lys Pro Gly Ala Ser Val Lys Val Ser Cys Lys Ala Ser Gly Phe Thr Phe Thr Ser Tyr Asp Ile Asn Trp Val Arg Gln Ala Pro Gly Gln Xaa3 Leu Glu Trp Met Gly Trp Ile Tyr Pro Gly Asp Xaa4 Ser Thr Lys Tyr Asn Glu Lys Phe Lys Gly Arg Val Thr Ile Thr Xaa5 Asp Xaa6 Ser Ala Xaa7 Thr Ala Tyr Met Glu Leu Xaa8 Ser Leu Arg Ser Glu Asp Thr Ala Val Tyr Xaa9 Cys Thr Arg Ser Phe Tyr Thr Met Asp Tyr Trp Gly Gln Gly Thr Thr Val Thr Val Ser Ser (SEQ ID NO: 56), or at least the CDRs thereof, wherein (a) Xaa1 is leucine (Leu) or phenylalanine (Phe), (b) Xaa2 is valine (Val), methionine (Met), or leucine (Leu), (c) Xaa3 is arginine (Arg) or glycine (Gly), (d) Xaa4 is glycine (Gly), serine (Ser), or alanine (Ala), (e) Xaa5 is arginine (Arg) or alanine (Ala), (f) Xaa6 is threonine (Thr) or lysine (Lys), (g) Xaa7 is serine (Ser) or asparagine (Asn), (h) Xaa8 is serine (Ser) or alanine (Ala), and (i) Xaa9 is tyrosine (Tyr) or phenylalanine (Phe). In some embodiments, the immunoglobulin heavy chain variable region comprises, consists of, or consists essentially of the amino acid sequence Gln Val Gln Xaa1 Xaa2 Gln Ser Gly Ala Glu Val Lys Lys Pro Gly Ala Ser Val Lys Val Ser Cys Lys Ala Ser Gly Phe Thr Phe Thr Ser Tyr Asp Ile Asn Trp Val Arg Gln Ala Pro Gly Gln Xaa3 Leu Glu Trp Met Gly Trp Ile Tyr Pro Gly Asp Xaa4 Ser Thr Lys Tyr Asn Glu Lys Phe Lys Gly Arg Val Thr Ile Thr Xaa5 Asp Xaa6 Ser Ala Ser Thr Ala Tyr Met Glu Leu Xaa7 Ser Leu Arg Ser Glu Asp Thr Ala Val Tyr Xaa8 Cys Thr Arg Ser Phe Tyr Thr Met Asp Tyr Trp Gly Gln Gly Thr Thr Val Thr Val Ser Ser (SEQ ID NO: 1), or at least the CDRs thereof, wherein (a) Xaa1 is leucine (Leu) or phenylalanine (Phe), (b) Xaa2 is valine (Val), methionine (Met), or leucine (Leu), (c) Xaa3 is arginine (Arg) or glycine (Gly), (d) Xaa4 is glycine (Gly), serine (Ser), or alanine (Ala), (e) Xaa5 is arginine (Arg) or alanine (Ala), (f) Xaa6 is threonine (Thr) or lysine (Lys), (g) Xaa7 is serine (Ser) or alanine (Ala), and (h) Xaa8 is tyrosine (Tyr) or phenylalanine (Phe).

The heavy chain variable region can comprise, consist of, or consist essentially of the amino acid sequence of SEQ ID NO: 56 or SEQ ID NO: 1, or at least the CDRs thereof, with any one of the aforementioned amino acid substitutions in any suitable combination. In one embodiment, the immunoglobulin heavy chain variable region comprises, consists of, or consists essentially of an amino acid sequence of any one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14, or at least the CDRs thereof.

In some embodiments, the IL-36R binding agent comprises: a CDR1 of the heavy chain variable region (HCDR1) comprising, consisting of, or consisting essentially of the amino acid sequence Phe Thr Phe Thr Ser Tyr Asp Ile Asn (SEQ ID NO: 59); a CDR2 of the heavy chain variable region (HCDR2) comprising, consisting of, or consisting essentially of the amino acid sequence of (a) Trp Ile Tyr Pro Gly Asp Gly Ser Thr Lys Tyr Asn Glu Lys Phe Lys Gly (SEQ ID NO: 60); (b) Trp Ile Tyr Pro Gly Asp Ser Ser Thr Lys Tyr Asn Glu Lys Phe Lys Gly (SEQ ID NO: 61); or (c) Trp Ile Tyr Pro Gly Asp Ala Ser Thr Lys Tyr Asn Glu Lys Phe Lys Gly (SEQ ID NO: 62); and/or a CDR3 of the heavy chain variable region (HCDR3) comprising, consisting of, or consisting essentially of the amino acid sequence Ser Phe Tyr Thr Met Asp Tyr (SEQ ID NO: 63).

In another embodiment, the immunoglobulin heavy chain variable region comprises, consists of, or consists essentially of the amino acid sequence Gln Val Gln Leu Val Gln Ser Gly Ala Glu Val Lys Lys Pro Gly Ala Ser Val Lys Val Ser Cys Lys Ala Ser Gly Tyr Thr Phe Thr Asn Tyr Xaa1 Met Xaa2 Trp Val Arg Gln Ala Pro Xaa3 Gln Gly Leu Glu Trp Met Gly Met Phe Xaa4 Pro Xaa5 Xaa6 Xaa7 Val Thr Arg Leu Asn Gln Lys Phe Lys Asp Arg Val Thr Met Thr Arg Asp Thr Ser Thr Ser Thr Val Tyr Met Glu Leu Ser Ser Leu Arg Ser Glu Asp Thr Ala Val Tyr Tyr Cys Ala Arg Thr Thr Ser Met Ile Ile Gly Gly Phe Ala Tyr Trp Gly Gln Gly Thr Leu Val Thr Val Ser Ser (SEQ ID NO: 15), or at least the CDRs thereof, wherein (a) Xaa1 is tryptophan (Trp) or tyrosine (Tyr), (b) Xaa2 is histidine (His), asparagine (Asn), or tyrosine (Tyr), (c) Xaa3 is glycine (Gly) or arginine (Arg), (d) Xaa4 is aspartic acid (Asp), glutamic acid (Glu), or histidine (His), (e) Xaa5 is serine (Ser), threonine (Thr), or tyrosine (Tyr), (f) Xaa6 is asparagine (Asn) or glycine (Gly), and (g) Xaa7 is serine (Ser), alanine (Ala), or aspartic acid (Asp).

The heavy chain variable region can comprise, consist of, or consist essentially of the amino acid sequence of SEQ ID NO: 15, or at least the CDRs thereof, with one of the aforementioned amino acid substitutions in any suitable combination. In one embodiment, the immunoglobulin heavy chain variable region comprises, consists of, or consists essentially of an amino acid sequence of any one of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24, or at least the CDRs thereof.

In one embodiment, the IL-36R binding agent comprises a heavy chain variable region comprising: an HCDR1 comprising, consisting of, or consisting essentially of the amino acid sequence selected from the group consisting of (a) Tyr Thr Phe Thr Asn Tyr Trp Met His (SEQ ID NO: 64); (b) Tyr Thr Phe Thr Asn Tyr Trp Met Asn (SEQ ID NO: 65); (c) Tyr Thr Phe Thr Asn Tyr Trp Met Tyr (SEQ ID NO: 66); and (d) Tyr Thr Phe Thr Asn Tyr Tyr Met Asn (SEQ ID NO: 67); an HCDR2 comprising, consisting of, or consisting essentially of the amino acid sequence selected from the group consisting of (a) Met Phe Asp Pro Ser Asn Ser Val Thr Arg Leu Asn Gln Lys Phe Lys Asp (SEQ ID NO: 68); (b) Met Phe Glu Pro Ser Asn Ala Val Thr Arg Leu Asn Gln Lys Phe Lys Asp (SEQ ID NO: 69); (c) Met Phe His Pro Ser Asn Ala Val Thr Arg Leu Asn Gln Lys Phe Lys Asp (SEQ ID NO: 70); and (d) Met Phe His Pro Thr Gly Asp Val Thr Arg Leu Asn Gln Lys Phe Lys Asp (SEQ ID NO: 71); and/or an HCDR3 comprising, consisting of, or consisting essentially of the amino acid sequence Thr Thr Ser Met Ile Ile Gly Gly Phe Ala Tyr (SEQ ID NO: 72).

In a further embodiment, the immunoglobulin heavy chain variable region comprises, consists of, or consists essentially of the amino acid sequence of Xaa1 Xaa2 Gln Xaa3 Gln Glu Ser Gly Pro Gly Leu Val Lys Pro Ser Gln Thr Leu Ser Leu Thr Cys Thr Val Xaa4 Xaa5 Tyr Ser Ile Thr Xaa6 Asp Phe Ala Trp Asn Trp Ile Arg Gln Xaa7 Pro Gly Xaa8 Xaa9 Leu Glu Trp Ile Gly Tyr Ile Ser Tyr Ser Gly Asp Thr Asn Tyr Asn Pro Ser Leu Lys Ser Arg Val Thr Ile Xaa10 Xaa11 Asp Thr Ser Lys Asn Gln Phe Ser Leu Lys Leu Ser Ser Val Thr Ala Ala Asp Thr Ala Xaa12 Tyr Xaa13 Cys Ala Ile Arg Gly Pro Tyr Ser Phe Thr Tyr Trp Gly Gln Gly Thr Leu Val Thr Val Ser Ser Xaa14 (SEQ ID NO: 57), or at least the CDRs thereof, wherein Xaa1 is glutamine (Gln) or aspartic acid (Asp); Xaa2 is valine (Val) or leucine (Leu); Xaa3 is leucine (Leu) or phenylalanine (Phe); Xaa4 is threonine (Thr) or serine (Ser); Xaa5 is glycine (Gly) or arginine (Arg); Xaa6 serine (Ser) or alanine (Ala); Xaa7 is proline (Pro) or phenylalanine (Phe); Xaa8 is lysine (Lys) or asparagine (Asn); Xaa9 is glycine (Gly) or lysine (Lys); Xaa10 is serine (Ser) or threonine (Thr); Xaa1 l is valine (Val) or arginine (Arg); Xaa12 is threonine (Thr) or valine (Val); Xaa13 is tyrosine (Tyr) or phenylalanine (Phe); and Xaa14 is alanine (Ala) or absent. In some embodiments, the heavy chain variable region comprises, consists of, or consists essentially of the amino acid sequence Xaa1 Val Gln Xaa2 Gln Glu Ser Gly Pro Gly Leu Val Lys Pro Ser Gln Thr Leu Ser Leu Thr Cys Thr Val Xaa3 Gly Tyr Ser Ile Thr Ser Asp Phe Ala Trp Asn Trp Ile Arg Gln Xaa4 Pro Gly Xaa5 Xaa6 Leu Glu Trp Ile Gly Tyr Ile Ser Tyr Ser Gly Asp Thr Asn Tyr Asn Pro Ser Leu Lys Ser Arg Val Thr Ile Xaa7 Xaa8 Asp Thr Ser Lys Asn Gln Phe Ser Leu Lys Leu Ser Ser Val Thr Ala Ala Asp Thr Ala Val Tyr Xaa9 Cys Ala Ile Arg Gly Pro Tyr Ser Phe Thr Tyr Trp Gly Gln Gly Thr Leu Val Thr Val Ser Ser (SEQ ID NO: 25), or at least the CDRs thereof, wherein (a) Xaa1 is glutamine (Gln) or aspartic acid (Asp), (b) Xaa2 is leucine (Leu) or phenylalanine (Phe), (c) Xaa3 is threonine (Thr) or serine (Ser), (d) Xaa4 is proline (Pro) or phenylalanine (Phe), (e) Xaa5 is lysine (Lys) or asparagine (Asn), (f) Xaa6 is glycine (Gly) or lysine (Lys), (g) Xaa7 is serine (Ser) or threonine (Thr), (h) Xaa8 is valine (Val) or arginine (Arg), and (i) Xaa9 is tyrosine (Tyr) or phenylalanine (Phe).

In some embodiments, the heavy chain variable region can comprise, consist of, or consist essentially of the amino acid sequence of SEQ ID NO: 57 or SEQ ID NO: 25, or at least the CDRs thereof, with one or more of the aforementioned amino acid substitutions in any suitable combination. In one embodiment, the immunoglobulin heavy chain variable region comprises, consists of, or consists essentially of an amino acid sequence of any one of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 51, SEQ ID NO: 52, SEQ ID NO: 53, or SEQ ID NO: 54, or at least the CDRs thereof.

In additional embodiments, the IL-36R binding agent may comprise an HCDR1 comprising, consisting of, or consisting essentially of the amino acid sequence selected from the group consisting of (a) Tyr Ser Ile Thr Ser Asp Phe Ala Trp Asn (SEQ ID NO: 73); and (b) Tyr Ser Ile Thr Ala Asp Phe Ala Trp Asn (SEQ ID NO: 74); an HCDR2 comprising, consisting of, or consisting essentially of the amino acid sequence Tyr Ile Ser Tyr Ser Gly Asp Thr Asn Tyr Asn Pro Ser Leu Lys Ser (SEQ ID NO: 75); and/or an HCDR3 comprising, consisting of, or consisting essentially of the amino acid sequence Arg Gly Pro Tyr Ser Phe Thr Tyr (SEQ ID NO: 76).

In another embodiment, the IL-36R binding agent comprises an immunoglobulin heavy chain variable region which comprises, consists of, or consists essentially of the amino acid sequence of SEQ ID NO: 33, SEQ ID NO: 34, or SEQ ID NO: 35, or at least the CDRs thereof.

In some embodiments, the immunoglobulin heavy chain variable region comprises an amino acid sequence that is at least 90% identical (e.g., at least 91%, at least 92%, at least 93%, at least 94%, at least 95%, at least 96%, at least 97%, at least 98%, at least 99%, or 100% identical) to any of the foregoing variable region sequences. Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (i.e., that are identical) as between the sequence of interest and the reference sequence divided by the length of the longest sequence (i.e., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FASTM, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

In addition to a heavy chain variable region as described herein, the IL-36R binding agent comprises an immunoglobulin light chain variable region that comprises, consists of, or consists essentially of the amino acid sequence Asp Ile Val Met Thr Gln Ser Pro Leu Ser Leu Pro Val Thr Pro Gly Glu Pro Ala Ser Ile Ser Cys Arg Ser Ser Lys Ser Leu Leu His Ser Asn Xaa1 Asn Thr Tyr Leu Tyr Trp Xaa2 Leu Gln Lys Pro Gly Gln Ser Pro Gln Leu Leu Ile Xaa3 Arg Met Ser Asn Leu Ala Ser Gly Val Pro Asp Arg Phe Ser Gly Ser Gly Ser Gly Thr Asp Phe Thr Leu Lys Ile Ser Arg Val Glu Ala Glu Asp Val Gly Val Tyr Tyr Cys Met Gln His Leu Glu Tyr Pro Phe Thr Phe Gly Gln Gly Thr Lys Leu Glu Ile Lys (SEQ ID NO: 36), or at least the CDRs thereof, wherein (a) Xaa1 is glycine (Gly) or alanine (Ala), (b) Xaa2 is phenylalanine (Phe) or tyrosine (Tyr), and (c) Xaa3 is tyrosine (Tyr) or serine (Ser).

The light chain variable region can comprise, consist of, or consist essentially of the amino acid sequence of SEQ ID NO: 36, or at least the CDRs thereof, with one or more of the aforementioned amino acid substitutions in any suitable combination. In one embodiment, the isolated immunoglobulin light chain variable region comprises, consists of, or consists essentially of an amino acid sequence of any one of SEQ ID NO: 37, SEQ ID NO: 38, or SEQ ID NO: 39, or at least the CDRs thereof.

In some embodiments, the IL-36R binding agent comprises a CDR1 of the light chain variable region (LCDR1) comprising, consisting of, or consisting essentially of the amino acid sequence selected of (a) Arg Ser Ser Lys Ser Leu Leu His Ser Asn Gly Asn Thr Tyr Leu Tyr (SEQ ID NO: 77); or (b) Arg Ser Ser Lys Ser Leu Leu His Ser Asn Ala Asn Thr Tyr Leu Tyr (SEQ ID NO: 78); a CDR2 of the light chain variable region (LCDR2) comprising, consisting of, or consisting essentially of the amino acid sequence Arg Met Ser Asn Leu Ala Ser (SEQ ID NO: 79); and a CDR3 of the light chain variable region (LCDR3) comprising, consisting of, or consisting essentially of the amino acid sequence Met Gln His Leu Glu Tyr Pro Phe Thr (SEQ ID NO: 80).

In some embodiments, the immunoglobulin light chain variable region comprises, consists of, or consists essentially of the amino acid sequence Asp Ile Val Met Thr Gln Thr Pro Leu Ser Leu Ser Val Thr Pro Gly Gln Pro Ala Ser Ile Ser Cys Arg Ser Ser Lys Ser Leu Leu His Xaa1 Asn Xaa2 Ile Thr Tyr Phe Tyr Trp Tyr Leu Xaa3 Lys Pro Gly Gln Pro Pro Gln Leu Leu Ile Tyr Gln Met Ser Asn Leu Ala Ser Gly Val Pro Asp Arg Phe Ser Gly Ser Gly Ser Gly Thr Asp Phe Thr Leu Lys Ile Ser Arg Val Glu Ala Glu Asp Val Gly Val Tyr Tyr Cys Ala Gln Asn Leu Glu Leu Pro Leu Thr Phe Gly Gly Gly Thr Lys Val Glu Ile Lys (SEQ ID NO: 40), or at least the CDRs thereof, wherein (a) Xaa1 is serine (Ser) or arginine (Arg), (b) Xaa2 is glycine (Gly) or alanine (Ala), and (c) Xaa3 is glutamine (GM) or histidine (His).

The light chain variable region can comprise, consist of, or consist essentially of the amino acid sequence of SEQ ID NO: 40, or at least the CDRs thereof, with the aforementioned amino acid substitutions in any combination. In one embodiment, the immunoglobulin light chain variable region polypeptide comprises, consists of, or consists essentially of an amino acid sequence of any one of SEQ ID NO: 41, SEQ ID NO: 42, SEQ ID NO: 43, or SEQ ID NO: 44; or at least the CDRs thereof.

In some embodiments, the light chain variable region comprises a LCDR1 comprising, consisting of, or consisting essentially of the amino acid sequence (a) Arg Ser Ser Lys Ser Leu Leu His Ser Asn Gly Ile Thr Tyr Phe Tyr (SEQ ID NO: 81); (b) Arg Ser Ser Lys Ser Leu Leu His Ser Asn Ala Ile Thr Tyr Phe Tyr (SEQ ID NO: 82); or (c) Arg Ser Ser Lys Ser Leu Leu His Arg Asn Ala Ile Thr Tyr Phe Tyr (SEQ ID NO: 83); a LCDR2 comprising, consisting of, or consisting essentially of the amino acid sequence Gln Met Ser Asn Leu Ala Ser (SEQ ID NO: 84); and a LCDR3 comprising, consisting of, or consisting essentially of the amino acid sequence Ala Gln Asn Leu Glu Leu Pro Leu Thr (SEQ ID NO: 85).

In further embodiments, the immunoglobulin light chain variable region comprises, consists of, or consists essentially of the amino acid sequence of Asp Ile Gln Met Thr Gln Ser Pro Ser Ser Leu Ser Ala Ser Val Gly Asp Arg Val Thr Ile Thr Cys Arg Ala Ser Gln Xaa1 Ile Asn Asn Tyr Leu Asn Trp Tyr Gln Gln Lys Pro Gly Lys Ala Pro Lys Leu Leu Ile Tyr Tyr Thr Ser Xaa2 Leu His Ser Gly Val Pro Ser Arg Phe Ser Xaa3 Ser Gly Ser Gly Xaa4 Asp Xaa5 Thr Phe Thr Ile Ser Ser Leu Gln Pro Glu Asp Ile Ala Thr Tyr Tyr Cys Gln Gln Gly His Thr Leu Pro Trp Thr Phe Gly Gly Gly Thr Lys Val Glu Ile Lys Xaa6 Xaa7 (SEQ ID NO: 58), or at least the CDRs thereof, wherein (a) Xaa1 is aspartic acid (Asp) or tryptophan (Trp), (b) Xaa2 is arginine (Arg) or methionine (Met), (c) Xaa3 is glycine (Gly), serine (Ser) or proline (Pro), (d) Xaa4 is threonine (Thr) or asparagines (Asn), (e) Xaa5 is phenylalanine (Phe) or tyrosine (Tyr), (f) Xaa6 is arginine (Arg) or absent, and (g) Xaa7 is threonine (Thr) or absent. In some embodiments, the immunoglobulin light chain variable region comprises, consists of, or consists essentially of the amino acid sequence Asp Ile Gln Met Thr Gln Ser Pro Ser Ser Leu Ser Ala Ser Val Gly Asp Arg Val Thr Ile Thr Cys Arg Ala Ser Gln Asp Ile Asn Asn Tyr Leu Asn Trp Tyr Gln Gln Lys Pro Gly Lys Ala Pro Lys Leu Leu Ile Tyr Tyr Thr Ser Arg Leu His Ser Gly Val Pro Ser Arg Phe Ser Xaa1 Ser Gly Ser Gly Thr Asp Xaa2 Thr Phe Thr Ile Ser Ser Leu Gln Pro Glu Asp Ile Ala Thr Tyr Tyr Cys Gln Gln Gly His Thr Leu Pro Trp Thr Phe Gly Gly Gly Thr Lys Val Glu Ile Lys (SEQ ID NO: 45), or at least the CDRs thereof, wherein (a) Xaa1 is serine (Ser) or proline (Pro), and (b) Xaa2 is phenylalanine (Phe) or tyrosine (Tyr).

The light chain variable region can comprise, consist of, or consist essentially of the amino acid sequence of SEQ ID NO: 58 or SEQ ID NO: 45, or at least the CDRs thereof, with one or more of the aforementioned amino acid substitutions in any suitable combination. In one embodiment, the immunoglobulin light chain variable region polypeptide comprises, consists of, or consists essentially of an amino acid sequence of any one of SEQ ID NO: 46, SEQ ID NO: 47, or SEQ ID NO: 55, or at least the CDRs thereof.

In some embodiments, the light chain variable region comprises an LCDR1 comprising, consisting of, or consisting essentially of the amino acid sequence (a) Arg Ala Ser Gln Asp Ile Asn Asn Tyr Leu Asn (SEQ ID NO: 86); or (b) Arg Ala Ser Gln Trp Ile Asn Asn Tyr Leu Asn (SEQ ID NO: 87); an LCDR2 comprising, consisting of, or consisting essentially of an amino acid sequence (a) Tyr Thr Ser Arg Leu His Ser (SEQ ID NO: 88); or (b) Tyr Thr Ser Met Leu His Ser (SEQ ID NO: 89); and an LCDR3 comprising, consisting of, or consisting essentially of the amino acid sequence Gln Gln Gly His Thr Leu Pro Trp Thr (SEQ ID NO: 90).

In another embodiment, the immunoglobulin light chain variable region comprises, consists of, or consists essentially of the amino acid sequence of SEQ ID NO: 48, SEQ ID NO: 49, or SEQ ID NO: 50, or at least the CDRs thereof.

In some embodiments, the immunoglobulin light chain variable region comprises an amino acid sequence that is at least 90% identical (e.g., at least 91%, at least 92%, at least 93%, at least 94%, at least 95%, at least 96%, at least 97%, at least 98%, at least 99%, or 100% identical) to any of the foregoing immunoglobulin light chain variable region sequences. Nucleic acid or amino acid sequence “identity” can be determined using the methods described herein.

As described above, the IL-36R binding agent can comprise an immunoglobulin heavy chain variable region and light chain variable region have any of the foregoing heavy and light chain variable region sequences, or the CDRs thereof. The CDR sequences can be the CDR sequences set forth herein or the CDR sequences as determined using any of several known methods (e.g., Kabat, Chothia, Martin (Enhanced Chothia), IGMT, or AHo).

In one embodiment, the IL-36R binding agent has an immunoglobulin heavy chain variable region comprising SEQ ID NO: 15 or SEQ ID NO: 22, or at least the CDR regions thereof; and an immunoglobulin light chain variable region comprising SEQ ID NO: 40 or SEQ ID NO: 44, or at least the CDR sequences thereof, wherein the CDRs are as determined with determined in accordance with any of the various known immunoglobulin numbering schemes, particularly in accordance with Kabat, Chothia, Martin (Enhanced Chothia), IGMT, or AHo. For example, in some embodiments, the antibody comprises a heavy chain variable region of SEQ ID NO: 22 and light chain variable region of SEQ ID NO: 44, or at least the CDRs thereof as determined by Kabat. In some embodiments, the antibody comprises a heavy chain variable region of SEQ ID NO: 22 and light chain variable region of SEQ ID NO: 44, or at least the CDRs thereof as determined by Chothia. In some embodiments, the antibody comprises a heavy chain variable region of SEQ ID NO: 22 and light chain variable region of SEQ ID NO: 44, or at least the CDRs thereof as determined by Martin. In some embodiments, the antibody comprises a heavy chain variable region of SEQ ID NO: 22 and light chain variable region of SEQ ID NO: 44, or at least the CDRs thereof as determined by IGMT. In some embodiments, the antibody comprises a heavy chain variable region of SEQ ID NO: 22 and light chain variable region of SEQ ID NO: 44, or at least the CDRs thereof as determined by AHo.

In some embodiments, the IL-36R binding agent comprises a heavy chain variable region comprising: an HCDR1 comprising, consisting of, or consisting essentially of the amino acid sequence selected from the group consisting of (a) Tyr Thr Phe Thr Asn Tyr Trp Met His (SEQ ID NO: 64); (b) Tyr Thr Phe Thr Asn Tyr Trp Met Asn (SEQ ID NO: 65); (c) Tyr Thr Phe Thr Asn Tyr Trp Met Tyr (SEQ ID NO: 66); and (d) Tyr Thr Phe Thr Asn Tyr Tyr Met Asn (SEQ ID NO: 67); an HCDR2 comprising, consisting of, or consisting essentially of the amino acid sequence selected from the group consisting of (a) Met Phe Asp Pro Ser Asn Ser Val Thr Arg Leu Asn Gln Lys Phe Lys Asp (SEQ ID NO: 68); (b) Met Phe Glu Pro Ser Asn Ala Val Thr Arg Leu Asn Gln Lys Phe Lys Asp (SEQ ID NO: 69); (c) Met Phe His Pro Ser Asn Ala Val Thr Arg Leu Asn Gln Lys Phe Lys Asp (SEQ ID NO: 70); and (d) Met Phe His Pro Thr Gly Asp Val Thr Arg Leu Asn Gln Lys Phe Lys Asp (SEQ ID NO: 71); and/or an HCDR3 comprising, consisting of, or consisting essentially of the amino acid sequence Thr Thr Ser Met Ile Ile Gly Gly Phe Ala Tyr (SEQ ID NO: 72); and comprises a light chain variable region comprising a LCDR1 comprising, consisting of, or consisting essentially of the amino acid sequence (a) Arg Ser Ser Lys Ser Leu Leu His Ser Asn Gly Ile Thr Tyr Phe Tyr (SEQ ID NO: 81); (b) Arg Ser Ser Lys Ser Leu Leu His Ser Asn Ala Ile Thr Tyr Phe Tyr (SEQ ID NO: 82); or (c) Arg Ser Ser Lys Ser Leu Leu His Arg Asn Ala Ile Thr Tyr Phe Tyr (SEQ ID NO: 83); a LCDR2 comprising, consisting of, or consisting essentially of the amino acid sequence Gln Met Ser Asn Leu Ala Ser (SEQ ID NO: 84); and a LCDR3 comprising, consisting of, or consisting essentially of the amino acid sequence Ala Gln Asn Leu Glu Leu Pro Leu Thr (SEQ ID NO: 85).

In a particular embodiment, the IL-36R binding agent comprises a heavy chain variable region comprising: an HCDR1 comprising, consisting of, or consisting essentially of the amino acid sequence selected from the group consisting of Tyr Thr Phe Thr Asn Tyr Trp Met Asn (SEQ ID NO: 65); an HCDR2 comprising, consisting of, or consisting essentially of the amino acid sequence selected from the group consisting of Met Phe His Pro Thr Gly Asp Val Thr Arg Leu Asn Gln Lys Phe Lys Asp (SEQ ID NO: 71); and/or an HCDR3 comprising, consisting of, or consisting essentially of the amino acid sequence Thr Thr Ser Met Ile Ile Gly Gly Phe Ala Tyr (SEQ ID NO: 72); and comprises a light chain variable region comprising a LCDR1 comprising, consisting of, or consisting essentially of the amino acid sequence Arg Ser Ser Lys Ser Leu Leu His Arg Asn Ala Ile Thr Tyr Phe Tyr (SEQ ID NO: 83); a LCDR2 comprising, consisting of, or consisting essentially of the amino acid sequence Gln Met Ser Asn Leu Ala Ser (SEQ ID NO: 84); and a LCDR3 comprising, consisting of, or consisting essentially of the amino acid sequence Ala Gln Asn Leu Glu Leu Pro Leu Thr (SEQ ID NO: 85).

Furthermore, the IL-36R binding agent can comprise an immunoglobulin heavy chain variable region and light chain variable region having specified percent identities to the heavy and light chain variable region sequences, such as at least 90% identical (e.g., at least 91%, at least 92%, at least 93%, at least 94%, at least 95%, at least 96%, at least 97%, at least 98%, at least 99%, or 100% identical). In some embodiments, the variance in sequence occurs outside the CDRs (as determined by any known method including Kabat, Chothia, Martin (Enhanced Chothia), IGMT, or AHo), such that the heavy and light chain sequences having the specified sequence identity to the specific sequences set forth herein retain the CDRs of such sequences. In an embodiment, the IL-36R binding agent comprises an immunoglobulin heavy chain variable region that is at least 90% identical (e.g., at least 91%, at least 92%, at least 93%, at least 94%, at least 95%, at least 96%, at least 97%, at least 98%, at least 99%, or 100% identical) to SEQ ID NO: 15 or SEQ ID NO: 22, optionally wherein the sequence retains the CDRs of SEQ ID NO: 15 or SEQ ID NO: 22; and comprises an immunoglobulin heavy chain variable region that is at least 90% identical (e.g., at least 91%, at least 92%, at least 93%, at least 94%, at least 95%, at least 96%, at least 97%, at least 98%, at least 99%, or 100% identical) to SEQ ID NO: 40 or SEQ ID NO: 44, optionally wherein the sequence retains the CDRs of SEQ ID NO: 40 or SEQ ID NO: 44; wherein the CDRs are as determined in accordance with any of the various known immunoglobulin numbering schemes, particularly in accordance with Kabat, Chothia, Martin (Enhanced Chothia), IGMT, or AHo. In a particular embodiment, the IL-36R binding agent comprises an immunoglobulin heavy chain variable region that is at least 90% identical (e.g., at least 91%, at least 92%, at least 93%, at least 94%, at least 95%, at least 96%, at least 97%, at least 98%, at least 99%, or 100% identical) to SEQ ID NO: 22, optionally wherein the sequence retains the CDRs of SEQ ID NO: 22; and comprises an immunoglobulin heavy chain variable region that is at least 90% identical (e.g., at least 91%, at least 92%, at least 93%, at least 94%, at least 95%, at least 96%, at least 97%, at least 98%, at least 99%, or 100% identical) to SEQ ID NO: 44, optionally wherein the sequence retains the CDRs of SEQ ID NO: 44; wherein the CDRs are as determined in accordance with any of the various known immunoglobulin numbering schemes, particularly in accordance with Kabat, Chothia, Martin (Enhanced Chothia), IGMT, or AHo.

Variation in sequence identity can be accomplished through addition, substitution, or deletion of one or more amino acid residues. An amino acid “replacement” or “substitution” refers to the replacement of one amino acid at a given position or residue by another amino acid at the same position or residue within a polypeptide sequence. The amino acid replacement or substitution can be conservative, semi-conservative, or non-conservative depending upon whether the substitution is by an amino acid residue that has similar properties to the residue being replaced. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Amino acids can be broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg).

Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine.

Aromatic amino acids may be sub-divided into two sub-groups: the “nitrogen ring sub-group” consisting of histidine and tryptophan and the “phenyl sub-group” consisting of phenylalanine and tyrosine.

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed herein, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

When the immunoglobulin light chain or heavy chain variable region “consists essentially of” any of the foregoing heavy or light chain variable region amino acid sequences, additional components can be included in the polypeptide that do not materially affect the polypeptide, such as those described herein. When the immunoglobulin light chain or heavy chain variable region “consists of”, the polypeptide does not comprise any additional components.

The IL-36R binding agent (e.g., antibody or antibody fragment) also can be a binding agent that competes with (e.g., binds to the same epitope or an overlapping epitope) an IL-36R binding agent comprising an immunoglobulin heavy chain variable region polypeptide or light chain variable region polypeptide described herein for binding to IL-36R (e.g., a binding agent comprising an Ig heavy chain comprising SEQ ID NO: 22 and an Ig light chain of SEQ ID NO: 44). Antibody competition can be assayed using routine peptide competition assays which utilize ELISA, Western blot, or immunohistochemistry methods (see, e.g., U.S. Pat. Nos. 4,828,981 and 8,568,992; and Braitbard et al., Proteome Sci., 4: 12 (2006)).

The IL-36R-binding agent can have any suitable affinity to IL-36R or an epitope thereof. The term “affinity” refers to the equilibrium constant for the reversible binding of two agents and is expressed as the dissociation constant (K_{D}). Affinity of a binding agent to a ligand, such as affinity of an antibody for an epitope, can be, for example, from about 1 picomolar (pM) to about 100 micromolar (μM) (e.g., from about 1 picomolar (pM) to about 1 nanomolar (nM), from about 1 nM to about 1 micromolar (μM), or from about 1 μM to about 100 μM). In one embodiment, the IL-36R-binding agent can bind to human IL-36R protein with a K_{D} less than or equal to 1 nanomolar (e.g., 0.9 nM, 0.8 nM, 0.7 nM, 0.6 nM, 0.5 nM, 0.4 nM, 0.3 nM, 0.2 nM, 0.1 nM, 0.05 nM, 0.025 nM, 0.01 nM, 0.001 nM, or a range defined by any two of the foregoing values). In another embodiment, the IL-36R-binding agent can bind to human IL-36R with a K_{D} less than or equal to 200 pM (e.g., 190 pM, 175 pM, 150 pM, 125 pM, 110 pM, 100 pM, 90 pM, 80 pM, 75 pM, 60 pM, 50 pM, 40 pM, 30 pM, 25 pM, 20 pM, 15 pM, 10 pM, 5 pM, 1 pM, or a range defined by any two of the foregoing values). In certain embodiments, the IL-36R binding agent cross-reacts with and inhibits both human and non-human primate (e.g., cynomolgus) IL-36R. Immunoglobulin affinity for an antigen or epitope of interest can be measured using any art-recognized assay. Such methods include, for example, fluorescence activated cell sorting (FACS), separable beads (e.g., magnetic beads), surface plasmon resonance (SPR), solution phase competition (KINEXA™), antigen panning, competitive binding assays, and/or ELISA (see, e.g., Janeway et al. (eds.), Immunobiology, 5th ed., Garland Publishing, New York, NY, 2001).

The IL-36R binding agent should have suitable stability in vivo. In one embodiment of the invention, the IL-36R-binding agent (e.g., an antibody) has an in vivo half-life between about 30 minutes and 45 days (e.g., about 30 minutes, about 45 minutes, about 1 hour, about 2 hours, about 4 hours, about 6 hours, about 10 hours, about 12 hours, about 1 day, about 5 days, about 10 days, about 15 days, about 25 days, about 35 days, about 40 days, about 45 days, or a range defined by any two of the foregoing values). In another embodiment, the IL-36R-binding agent has an in vivo half-life between about 2 hours and 20 days (e.g., about 5 hours, about 10 hours, about 15 hours, about 20 hours, about 2 days, about 3 days, about 7 days, about 12 days, about 14 days, about 17 days, about 19 days, or a range defined by any two of the foregoing values). In another embodiment, the IL-36R-binding agent has an in vivo half-life between about 10 days and about 40 days (e.g., about 10 days, about 13 days, about 16 days, about 18 days, about 20 days, about 23 days, about 26 days, about 29 days, about 30 days, about 33 days, about 37 days, about 38 days, about 39 days, about 40 days, or a range defined by any two of the foregoing values).

The stability of the IL-36R-binding agent described herein also can be measured in terms of the transition mid-point value (Tₘ), which is the temperature where 50% of the amino acid sequence is in its native confirmation, and the other 50% is denatured. In general, the higher the Tₘ, the more stable the protein. In one embodiment of the invention, the IL-36R-binding agent comprises a transition mid-point value (Tₘ) in vitro of about 60-100° C. For example, the IL-36R-binding agent can comprise a Tₘ in vitro of about 65-80° C. (e.g., 66° C., 68° C., 70° C., 71° C., 75° C., or 79° C.), about 80-90° C. (e.g., about 81° C., 85° C., or 89° C.), or about 90-100° C. (e.g., about 91° C., about 95° C., or about 99° C.).

The stability of the IL-36R-binding agent can be measured using any other suitable assay known in the art, such as, for example, measuring serum half-life, differential scanning calorimetry (DSC), thermal shift assays, and pulse-chase assays. Other methods of measuring protein stability in vivo and in vitro that can be used in the context of the invention are described in, for example, Protein Stability and Folding, B. A. Shirley (ed.), Human Press, Totowa, New Jersey (1995); Protein Structure, Stability, and Interactions (Methods in Molecular Biology), Shiver J. W. (ed.), Humana Press, New York, NY (2010); and Ignatova, Microb. Cell Fact., 4: 23 (2005).

The IL-36R-binding agent desirably exhibits one or more of the following biological activities: (a) inhibits the interaction between IL-36R and IL-36α, IL-36β, and/or IL-36γ, (b) inhibits intracellular signaling mediated by IL-36R, and/or (c) cross-reacts with and inhibits the activity of human and non-human primate (e.g., cynomolgus) IL-36R. Other biological properties or characteristics of an antigen-binding agent recognized in the art include, for example, avidity, selectivity, solubility, folding, immunotoxicity, expression, and formulation. The aforementioned properties or characteristics can be observed, measured, and/or assessed using standard techniques including, but not limited to, ELISA, competitive ELISA, surface plasmon resonance analysis (BIACORE™), or KINEXA™, in vitro or in vivo neutralization assays, receptor-ligand binding assays, cytokine or growth factor production and/or secretion assays, and signal transduction and immunohistochemistry assays.

The terms “inhibit” or “neutralize,” as used herein with respect to the activity of the IL-36 pathway inhibitor, e.g., the IL-36R-binding agent, refer to the ability to substantially antagonize, prohibit, prevent, restrain, slow, disrupt, alter, eliminate, stop, or reverse the progression or severity of, for example, the biological activity of an IL-36 cytokine or IL-36R, or a disease or condition associated with an IL-36 cytokine or IL-36R, e.g., hidradenitis suppurativa. For example, the IL-36R-binding agent preferably inhibits or neutralizes the activity of IL-36R by at least about 20%, about 30%, about 40%, about 50%, about 60%, about 70%, about 80%, about 90%, about 95%, about 100%, or a range defined by any two of the foregoing values.

The IL-36R-binding agent of the present inventive method can be a whole antibody, as described herein, or an antibody fragment. The terms “fragment of an antibody,” “antibody fragment,” and “functional fragment of an antibody” are used interchangeably herein to mean one or more fragments of an antibody that retain the ability to specifically bind to an antigen (see, generally, Holliger et al., Nat. Biotech., 23(9): 1126-1129 (2005)). The IL-36R-binding agent can contain any IL-36R-binding antibody fragment. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (VH or VL) polypeptide that specifically binds antigen.

When the IL-36R-binding agent is an antibody or antibody fragment, the antibody or antibody fragment can comprise a heavy chain constant region (Fe) of any suitable class. In some embodiments, the antibody or antibody fragment comprises a heavy chain constant region that is based upon wild-type IgG1, IgG2, or IgG4 antibodies, or variants thereof. It will be appreciated that each antibody class, or isotype, engages a distinct set of effector mechanisms for disposing of or neutralizing antigen once recognized. As such, in some embodiments, when the IL-36R-binding agent is an antibody or antibody fragment, it can exhibit one or more effector functions, such as participation in antibody-dependent complement-mediated lysis or antibody-dependent cellular toxicity via interactions with effector molecules and cells (e.g., activation of the complement system).

The IL-36R-binding agent also can be a single chain antibody fragment. Examples of single chain antibody fragments include, but are not limited to, (i) a single chain Fv (scFv), which is a monovalent molecule consisting of the two domains of the Fv fragment (i.e., V_{L} and V_{H}) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (ii) a diabody, which is a dimer of polypeptide chains, wherein each polypeptide chain comprises a V_{H} connected to a V_{L} by a peptide linker that is too short to allow pairing between the V_{H} and V_{L} on the same polypeptide chain, thereby driving the pairing between the complementary domains on different V_{H}−V_{L} polypeptide chains to generate a dimeric molecule having two functional antigen binding sites. Antibody fragments are known in the art and are described in more detail in, e.g., U.S. Patent Application Publication 2009/0093024 A1.

The IL-36R-binding agent also can be an intrabody or fragment thereof. An intrabody is an antibody which is expressed and which functions intracellularly. Intrabodies typically lack disulfide bonds and are capable of modulating the expression or activity of target genes through their specific binding activity. Intrabodies include single domain fragments such as isolated V_{H} and V_{L} domains and scFvs. An intrabody can include sub-cellular trafficking signals attached to the N or C terminus of the intrabody to allow expression at high concentrations in the sub-cellular compartments where a target protein is located. Upon interaction with a target gene, an intrabody modulates target protein function and/or achieves phenotypic/functional knockout by mechanisms such as accelerating target protein degradation and sequestering the target protein in a non-physiological sub-cellular compartment. Other mechanisms of intrabody-mediated gene inactivation can depend on the epitope to which the intrabody is directed, such as binding to the catalytic site on a target protein or to epitopes that are involved in protein-protein, protein-DNA, or protein-RNA interactions.

The IL-36R-binding agent also can be an antibody conjugate. In this respect, the IL-36R-binding agent can be a conjugate of (1) an antibody, an alternative scaffold, or fragments thereof, and (2) a protein or non-protein moiety comprising the IL-36R-binding agent. For example, the IL-36R-binding agent can be all or part of an antibody conjugated to a peptide, a fluorescent molecule, or a chemotherapeutic agent.

The IL-36R-binding agent can be, or can be obtained from, a human antibody, a non-human antibody, or a chimeric antibody. A “chimeric” antibody is an antibody or fragment thereof comprising both human and non-human regions. Preferably, the IL-36R-binding agent is a humanized antibody. A “humanized” antibody is a monoclonal antibody comprising a human antibody scaffold and at least one CDR obtained or derived from a non-human antibody. Non-human antibodies include antibodies isolated from any non-human animal, such as, for example, a rodent (e.g., a mouse or rat). A humanized antibody can comprise, one, two, or three CDRs obtained or derived from a non-human antibody. In one embodiment of the invention, CDRH3 of the IL-36R-binding agent is obtained or derived from a mouse monoclonal antibody, while the remaining variable regions and constant region of the IL-36R-binding agent are obtained or derived from a human monoclonal antibody.

A human antibody, a non-human antibody, a chimeric antibody, or a humanized antibody can be obtained by any means, including via in vitro sources (e.g., a hybridoma or a cell line producing an antibody recombinantly) and in vivo sources (e.g., rodents). Methods for generating antibodies are known in the art and are described in, for example, Köhler and Milstein, Eur. J. Immunol., 5: 511-519 (1976); Harlow and Lane (eds.), Antibodies: A Laboratory Manual, CSH Press (1988); and Janeway et al. (eds.), Immunobiology, 5th Ed., Garland Publishing, New York, NY (2001)). In certain embodiments, a human antibody or a chimeric antibody can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the Medarex HUMAB-MOUSE™, the Kirin TC MOUSE™, and the Kyowa Kirin KM-MOUSE™ (see, e.g., Lonberg, Nat. Biotechnol., 23(9): 1117-25 (2005), and Lonberg, Handb. Exp. Pharmacol., 181: 69-97 (2008)). A humanized antibody can be generated using any suitable method known in the art (see, e.g., An, Z. (ed.), Therapeutic Monoclonal Antibodies: From Bench to Clinic, John Wiley & Sons, Inc., Hoboken, New Jersey (2009)), including, e.g., grafting of non-human CDRs onto a human antibody scaffold (see, e.g., Kashmiri et al., Methods, 36(1): 25-34 (2005); and Hou et al., J. Biochem., 144(1): 115-120 (2008)). In one embodiment, a humanized antibody can be produced using the methods described in, e.g., U.S. Patent Application Publication 2011/0287485 A1.

In one embodiment, a CDR (e.g., CDR1, CDR2, or CDR3) or a variable region of the immunoglobulin heavy chain variable region and/or the immunoglobulin light chain variable region described herein can be transplanted (i.e., grafted) into another molecule, such as an antibody or non-antibody polypeptide, using either protein chemistry or recombinant DNA technology. In this regard, the invention encompasses an IL-36R-binding agent comprising at least one CDR of an immunoglobulin heavy chain and/or light chain variable region as described herein. The IL-36R-binding agent can comprise one, two, or three CDRs of an immunoglobulin heavy chain and/or light chain variable region as described herein.

The IL-36R binding agent can be formulated as a composition for administration to a subject (e.g., a mammal, such as a human or non-human primate) by any suitable route of administration, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. In some embodiments, the composition is administered to a mammal using peripheral systemic delivery by intravenous or subcutaneous injection.

The composition can comprise the IL-36R binding agent and a suitable carrier such as are well known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

The IL-36R binding agent can be used at dosages and for periods of time necessary, to achieve a desired pharmacologic and/or physiologic effect (“therapeutically effective amount”). The therapeutically effective amount may vary according to factors such as the disease state, age, sex, and weight of the individual, and the ability of the IL-36 pathway inhibitor, e.g., the IL-36R-binding agent, to elicit a desired response in the individual. For example, a therapeutically effective amount of an inhibitor of the IL-36 pathway can an amount that decreases the bioactivity of any one of the IL-36 cytokines and/or IL-36R, and thus decreases the activity of the IL-36 signalling pathway. In some embodiments, the dose is in the range of 1 pg/kg to 20 mg/kg of animal or human body weight; however, doses below or above this exemplary range are within the scope of the invention. The daily parenteral dose can be about 0.00001 μg/kg to about 20 mg/kg of total body weight (e.g., about 0.001 μg/kg, about 0.1 μg/kg, about 1 μg/kg, about 5 μg/kg, about 10 μg/kg, about 100 lag/kg, about 500 μg/kg, about 1 mg/kg, about 5 mg/kg, about 10 mg/kg, or a range defined by any two of the foregoing values), preferably from about 0.1 μg/kg to about 10 mg/kg of total body weight (e.g., about 0.5 μg/kg, about 1 μg/kg, about 50 μg/kg, about 150 μg/kg, about 300 μg/kg, about 750 μg/kg, about 1.5 mg/kg, about 5 mg/kg, or a range defined by any two of the foregoing values), more preferably from about 1 μg/kg to 5 mg/kg of total body weight (e.g., about 3 μg/kg, about 15 μg/kg, about 75 μg/kg, about 300 μg/kg, about 900 μg/kg, about 2 mg/kg, about 4 mg/kg, or a range defined by any two of the foregoing values), and even more preferably from about 0.5 to 15 mg/kg body weight per day (e.g., about 1 mg/kg, about 2.5 mg/kg, about 3 mg/kg, about 6 mg/kg, about 9 mg/kg, about 11 mg/kg, about 13 mg/kg, or a range defined by any two of the foregoing values).

In some embodiments the dose is about 20 mg or more (e.g., about 30 mg or more, about 50 mg or more, about 75 mg or more, or about 100 mg or more), and about 1000 mg or less (e.g., about 900 mg or less, about 800 mg or less, about 700 mg or less, about 600 mg or less, about 500 mg or less, about 400 mg or less, or about 300 mg or less) every 1-6 weeks (e.g., every week, every two weeks, every three weeks or every four weeks). In some embodiments, the IL-36 inhibitor is administered in a single loading does of about 1.5×-10× (e.g., about 2×-8×) the amount of following maintenance doses. Thus, for instance, the IL-36R binding agent can be administered in a loading dose of about 200 mg-750 mg (e.g., about 300 mg-500 mg, or about 350 mg-450 mg) followed by a maintenance dose of about 50-300 mg (e.g., about 100 mg-300 mg or about 150 mg-250 mg) every two weeks or every month or two months (e.g., every 2-8 weeks or 2-4 weeks) thereafter as needed to effect or maintain a therapeutic response.

In some embodiments, the IL-36R binding agent provides a long-lasting effect against hidradenitis suppurativa and allows for relatively infrequent dosing, for instance, on a schedule of not more than once every 14 days, 21 days, or 30 days. In some embodiments, an even longer interval can be used (e.g., not more than once every 45 days, 60 days, 90 days, or 120 days).

Therapeutic efficacy can be monitored by periodic assessment of treated patients. For repeated administrations over several days or longer, depending on the condition, the treatment can be repeated until a desired suppression of hidradenitis suppurativa occurs, or alternatively, the treatment can be continued for the lifetime of the patient. However, other dosage regimens may be useful and are within the scope of the invention. The desired dosage can be delivered by a single bolus administration of the IL-36R binding agent or composition described herein, by multiple bolus administrations of the IL-36R binding agent composition described herein, or by continuous infusion administration of the IL-36R binding agent or composition described herein.

The IL-36R-binding agent of the invention may be administered alone or in combination with other drugs. For example, the IL-36R-binding agent can be administered in combination with other agents for the treatment or prevention of the diseases disclosed herein, such as an anti-inflammatory agent including, for example, corticosteroids (e.g., prednisone and fluticasone), non-steroidal anti-inflammatory drugs (NSAIDs) (e.g., aspirin, ibuprofen, and naproxen), biologics (e.g., infliximab (REMICADE™), adalimumab (HUMIRA™), or etanercept (ENBREL™)), methotrexate (MTX), an oral retinoid (e.g. acitretin (SORIATANE™)), topical steroids, anti-infectives and/or antibiotics, or other agents useful for alleviating the severity of hidradenitis suppurativa symptoms.

The invention further encompasses an IL-36R binding agent or composition comprising same, as described herein, for use to treat hidradenitis suppurativa in accordance with the methods provided herein. In some embodiments, the IL-36R binding agent comprises an immunoglobulin heavy chain variable region of SEQ ID NO: 22 or at least the CDRs thereof, and an immunoglobulin light chain variable region of SEQ ID NO: 44 or at least the CDRs thereof, as described herein.

The following examples further illustrate the invention but, of course, should not be construed as in any way limiting its scope.

### Example 1

Gene expression of IL-36α, IL-36β, IL-36γ, and IL-36Ra was measured in samples from skin lesions of patients with clinically confirmed hidradenitis suppurativa, and the expression levels compared to the expression levels found in normal skin from healthy subjects. Expression levels of all four biomarkers were increased in the hidradenitis suppurativa patients. In particular, IL-36γ expression levels were found to be more than ten-fold higher in the hidradenitis suppurativa patient samples than in normal samples, and IL-36α and IL-36β levels in the hidradenitis suppurativa patient samples were found to be approximately 5-fold higher than in normal samples (data normalized to the housekeeping gene 18S).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating hidradenitis suppurativa in a subject, the method comprising inhibiting IL-36 signaling in the subject, thereby treating the hidradenitis suppurativa.

2. The method of claim 1, wherein the subject is a mammal.

3. The method of claim 1, wherein the mammal is a human.

4. The method of claim 1, wherein the subject has increased expression of at least one of an interleukin-36 (IL-36) cytokine, interleukin-36 receptor (IL-36R), or mRNA encoding same in skin affected by the hidradenitis suppurativa.

5. The method of claim 4, wherein the wherein the subject has increased expression of IL-36γ in skin affected by the hidradenitis suppurativa.

6. The method of claim 1, wherein IL-36 signaling is inhibited by administering to the subject an IL-36R-binding agent.

7. The method of claim 6, wherein the IL-36R-binding agent comprises an immunoglobulin heavy chain polypeptide and immunoglobulin light chain polypeptide, wherein the immunoglobulin heavy chain polypeptide comprises:
Gln Val Gln Xaa1 Xaa2 Gln Ser Gly Ala Glu Val Lys Lys Pro Gly Ala Ser Val Lys Val Ser Cys Lys Ala Ser Gly Phe Thr Phe Thr Ser Tyr Asp Ile Asn Trp Val Arg Gln Ala Pro Gly Gln Xaa3 Leu Glu Trp Met Gly Trp Ile Tyr Pro Gly Asp Xaa4 Ser Thr Lys Tyr Asn Glu Lys Phe Lys Gly Arg Val Thr Ile Thr Xaa5 Asp Xaa6 Ser Ala Xaa7 Thr Ala Tyr Met Glu Leu Xaa8 Ser Leu Arg Ser Glu Asp Thr Ala Val Tyr Xaa9 Cys Thr Arg Ser Phe Tyr Thr Met Asp Tyr Trp Gly Gln Gly Thr Thr Val Thr Val Ser Ser (SEQ ID NO: 56), wherein
(a) Xaa1 is leucine (Leu) or phenylalanine (Phe),
(b) Xaa2 is valine (Val), methionine (Met), or leucine (Leu),
(c) Xaa3 is arginine (Arg) or glycine (Gly),
(d) Xaa4 is glycine (Gly), serine (Ser), or alanine (Ala),
(e) Xaa5 is arginine (Arg) or alanine (Ala),
(f) Xaa6 is threonine (Thr) or lysine (Lys),
(g) Xaa7 is serine (Ser) or asparagine (Asn),
(h) Xaa8 is serine (Ser) or alanine (Ala), and
(i) Xaa9 is tyrosine (Tyr) or phenylalanine (Phe);
or
Gln Val Gln Xaa1 Xaa2 Gln Ser Gly Ala Glu Val Lys Lys Pro Gly Ala Ser Val Lys Val Ser Cys Lys Ala Ser Gly Phe Thr Phe Thr Ser Tyr Asp Ile Asn Trp Val Arg Gln Ala Pro Gly Gln Xaa3 Leu Glu Trp Met Gly Trp Ile Tyr Pro Gly Asp Xaa4 Ser Thr Lys Tyr Asn Glu Lys Phe Lys Gly Arg Val Thr Ile Thr Xaa5 Asp Xaa6 Ser Ala Ser Thr Ala Tyr Met Glu Leu Xaa7 Ser Leu Arg Ser Glu Asp Thr Ala Val Tyr Xaa8 Cys Thr Arg Ser Phe Tyr Thr Met Asp Tyr Trp Gly Gln Gly Thr Thr Val Thr Val Ser Ser (SEQ ID NO: 1), wherein
(a) Xaa1 is leucine (Leu) or phenylalanine (Phe),
(b) Xaa2 is valine (Val), methionine (Met), or leucine (Leu),
(c) Xaa3 is arginine (Arg) or glycine (Gly),
(d) Xaa4 is glycine (Gly), serine (Ser), or alanine (Ala),
(e) Xaa5 is arginine (Arg) or alanine (Ala),
(f) Xaa6 is threonine (Thr) or lysine (Lys),
(g) Xaa7 is serine (Ser) or alanine (Ala), and
(h) Xaa8 is tyrosine (Tyr) or phenylalanine (Phe);
or
Gln Val Gln Leu Val Gln Ser Gly Ala Glu Val Lys Lys Pro Gly Ala Ser Val Lys Val Ser Cys Lys Ala Ser Gly Tyr Thr Phe Thr Asn Tyr Xaa1 Met Xaa2 Trp Val Arg Gln Ala Pro Xaa3 Gln Gly Leu Glu Trp Met Gly Met Phe Xaa4 Pro Xaa5 Xaa6 Xaa7 Val Thr Arg Leu Asn Gln Lys Phe Lys Asp Arg Val Thr Met Thr Arg Asp Thr Ser Thr Ser Thr Val Tyr Met Glu Leu Ser Ser Leu Arg Ser Glu Asp Thr Ala Val Tyr Tyr Cys Ala Arg Thr Thr Ser Met Ile Ile Gly Gly Phe Ala Tyr Trp Gly Gln Gly Thr Leu Val Thr Val Ser Ser (SEQ ID NO: 15), wherein
(a) Xaa1 is tryptophan (Trp) or tyrosine (Tyr),
(b) Xaa2 is histidine (His), asparagine (Asn), or tyrosine (Tyr),
(c) Xaa3 is glycine (Gly) or arginine (Arg),
(d) Xaa4 is aspartic acid (Asp), glutamic acid (Glu), or histidine (His),
(e) Xaa5 is serine (Ser), threonine (Thr), or tyrosine (Tyr),
(f) Xaa6 is asparagine (Asn) or glycine (Gly), and
(g) Xaa7 is serine (Ser), alanine (Ala), or aspartic acid (Asp);
or
Xaa1 Xaa2 Gln Xaa3 Gln Glu Ser Gly Pro Gly Leu Val Lys Pro Ser Gln Thr Leu Ser Leu Thr Cys Thr Val Xaa4 Xaa5 Tyr Ser Ile Thr Xaa6 Asp Phe Ala Trp Asn Trp Ile Arg Gln Xaa7 Pro Gly Xaa8 Xaa9 Leu Glu Trp Ile Gly Tyr Ile Ser Tyr Ser Gly Asp Thr Asn Tyr Asn Pro Ser Leu Lys Ser Arg Val Thr Ile Xaa10 Xaa1 l Asp Thr Ser Lys Asn Gln Phe Ser Leu Lys Leu Ser Ser Val Thr Ala Ala Asp Thr Ala Xaa12 Tyr Xaa13 Cys Ala Ile Arg Gly Pro Tyr Ser Phe Thr Tyr Trp Gly Gln Gly Thr Leu Val Thr Val Ser Ser Xaa14 (SEQ ID NO: 57), wherein
(a) Xaa1 is glutamine (GM) or aspartic acid (Asp),
(b) Xaa2 is valine (Val) or leucine (Leu),
(c) Xaa3 is leucine (Leu) or phenylalanine (Phe),
(d) Xaa4 is threonine (Thr) or serine (Ser),
(e) Xaa5 is glycine (Gly) or arginine (Arg),
(f) Xaa6 serine (Ser) or alanine (Ala),
(g) Xaa7 is proline (Pro) or phenylalanine (Phe),
(h) Xaa8 is lysine (Lys) or asparagine (Asn),
(i) Xaa9 is glycine (Gly) or lysine (Lys),
(j) Xaa10 is serine (Ser) or threonine (Thr),
(k) Xaa11 is valine (Val) or arginine (Arg),
(l) Xaa12 is threonine (Thr) or valine (Val),
(m) Xaa13 is tyrosine (Tyr) or phenylalanine (Phe), and
(n) Xaa14 is alanine (Ala) or absent;
or
Xaa1 Val Gln Xaa2 Gln Glu Ser Gly Pro Gly Leu Val Lys Pro Ser Gln Thr Leu Ser Leu Thr Cys Thr Val Xaa3 Gly Tyr Ser Ile Thr Ser Asp Phe Ala Trp Asn Trp Ile Arg Gln Xaa4 Pro Gly Xaa5 Xaa6 Leu Glu Trp Ile Gly Tyr Ile Ser Tyr Ser Gly Asp Thr Asn Tyr Asn Pro Ser Leu Lys Ser Arg Val Thr Ile Xaa7 Xaa8 Asp Thr Ser Lys Asn Gln Phe Ser Leu Lys Leu Ser Ser Val Thr Ala Ala Asp Thr Ala Val Tyr Xaa9 Cys Ala Ile Arg Gly Pro Tyr Ser Phe Thr Tyr Trp Gly Gln Gly Thr Leu Val Thr Val Ser Ser (SEQ ID NO: 25), wherein
(a) Xaa1 is glutamine (GM) or aspartic acid (Asp),
(b) Xaa2 is leucine (Leu) or phenylalanine (Phe),
(c) Xaa3 is threonine (Thr) or serine (Ser),
(d) Xaa4 is proline (Pro) or phenylalanine (Phe),
(e) Xaa5 is lysine (Lys) or asparagine (Asn),
(f) Xaa6 is glycine (Gly) or lysine (Lys),
(g) Xaa7 is serine (Ser) or threonine (Thr),
(h) Xaa8 is valine (Val) or arginine (Arg), and
(i) Xaa9 is tyrosine (Tyr) or phenylalanine (Phe);
or
any one of SEQ ID NOs: 2-14, 16-24, 26-35, or 51-54; or at least CDR1, CDR2, and CDR3 thereof;
and wherein the immunoglobulin light chain polypeptide polypeptide comprises:
Asp Ile Val Met Thr Gln Ser Pro Leu Ser Leu Pro Val Thr Pro Gly Glu Pro Ala Ser Ile Ser Cys Arg Ser Ser Lys Ser Leu Leu His Ser Asn Xaa1 Asn Thr Tyr Leu Tyr Trp Xaa2 Leu Gln Lys Pro Gly Gln Ser Pro Gln Leu Leu Ile Xaa3 Arg Met Ser Asn Leu Ala Ser Gly Val Pro Asp Arg Phe Ser Gly Ser Gly Ser Gly Thr Asp Phe Thr Leu Lys Ile Ser Arg Val Glu Ala Glu Asp Val Gly Val Tyr Tyr Cys Met Gln His Leu Glu Tyr Pro Phe Thr Phe Gly Gln Gly Thr Lys Leu Glu Ile Lys (SEQ ID NO: 36), wherein
(a) Xaa1 is glycine (Gly) or alanine (Ala),
(b) Xaa2 is phenylalanine (Phe) or tyrosine (Tyr), and
(c) Xaa3 is tyrosine (Tyr) or serine (Ser);
or
(SEQ ID NO: 40)
Asp Ile Val Met Thr Gln Thr Pro Leu Ser Leu Ser
Val Thr Pro Gly Gln Pro Ala Ser Ile Ser Cys Arg
Ser Ser Lys Ser Leu Leu His Xaa1 Asn Xaa2 Ile Thr
Tyr Phe Tyr Trp Tyr Leu Xaa3 Lys Pro Gly Gln Pro
Pro Gln Leu Leu Ile Tyr Gln Met Ser Asn Leu Ala
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Ala Gln Asn
Leu Glu Leu Pro Leu Thr Phe Gly Gly Gly Thr Lys
Val Glu Ile Lys,
(a) Xaa1 is serine (Ser) or arginine (Arg),
(b) Xaa2 is glycine (Gly) or alanine (Ala), and
(c) Xaa3 is glutamine (GM) or histidine (His);
or
Asp Ile Gln Met Thr Gln Ser Pro Ser Ser Leu Ser Ala Ser Val Gly Asp Arg Val Thr Ile Thr Cys Arg Ala Ser Gln Xaa1 Ile Asn Asn Tyr Leu Asn Trp Tyr Gln Gln Lys Pro Gly Lys Ala Pro Lys Leu Leu Ile Tyr Tyr Thr Ser Xaa2 Leu His Ser Gly Val Pro Ser Arg Phe Ser Xaa3 Ser Gly Ser Gly Xaa4 Asp Xaa5 Thr Phe Thr Ile Ser Ser Leu Gln Pro Glu Asp Ile Ala Thr Tyr Tyr Cys Gln Gln Gly His Thr Leu Pro Trp Thr Phe Gly Gly Gly Thr Lys Val Glu Ile Lys Xaa6 Xaa7 (SEQ ID NO: 58), wherein
(a) Xaa1 is aspartic acid (Asp) or tryptophan (Trp),
(b) Xaa2 is arginine (Arg) or methionine (Met),
(c) Xaa3 is glycine (Gly), serine (Ser) or proline (Pro),
(d) Xaa4 is threonine (Thr) or asparagines (Asn),
(e) Xaa5 is phenylalanine (Phe) or tyrosine (Tyr),
(f) Xaa6 is arginine (Arg) or absent, and
(g) Xaa7 is threonine (Thr) or absent;
or
Asp Ile Gln Met Thr Gln Ser Pro Ser Ser Leu Ser Ala Ser Val Gly Asp Arg Val Thr Ile Thr Cys Arg Ala Ser Gln Asp Ile Asn Asn Tyr Leu Asn Trp Tyr Gln Gln Lys Pro Gly Lys Ala Pro Lys Leu Leu Ile Tyr Tyr Thr Ser Arg Leu His Ser Gly Val Pro Ser Arg Phe Ser Xaa1 Ser Gly Ser Gly Thr Asp Xaa2 Thr Phe Thr Ile Ser Ser Leu Gln Pro Glu Asp Ile Ala Thr Tyr Tyr Cys Gln Gln Gly His Thr Leu Pro Trp Thr Phe Gly Gly Gly Thr Lys Val Glu Ile Lys (SEQ ID NO: 45), wherein
(a) Xaa1 is serine (Ser) or proline (Pro), and
(b) Xaa2 is phenylalanine (Phe) or tyrosine (Tyr);
or comprises any one of SEQ ID NO: 37-39, 41-44, 46-50, or 55; or at least CDR1, CDR2, and CDR3 thereof.

8. The method of claim 7, wherein the immunoglobulin heavy chain polypeptide of the IL-36R-binding agent comprises:
Gln Val Gln Leu Val Gln Ser Gly Ala Glu Val Lys Lys Pro Gly Ala Ser Val Lys Val Ser Cys Lys Ala Ser Gly Tyr Thr Phe Thr Asn Tyr Xaa1 Met Xaa2 Trp Val Arg Gln Ala Pro Xaa3 Gln Gly Leu Glu Trp Met Gly Met Phe Xaa4 Pro Xaa5 Xaa6 Xaa7 Val Thr Arg Leu Asn Gln Lys Phe Lys Asp Arg Val Thr Met Thr Arg Asp Thr Ser Thr Ser Thr Val Tyr Met Glu Leu Ser Ser Leu Arg Ser Glu Asp Thr Ala Val Tyr Tyr Cys Ala Arg Thr Thr Ser Met Ile Ile Gly Gly Phe Ala Tyr Trp Gly Gln Gly Thr Leu Val Thr Val Ser Ser (SEQ ID NO: 15), wherein
(a) Xaa1 is tryptophan (Trp) or tyrosine (Tyr),
(b) Xaa2 is histidine (His), asparagine (Asn), or tyrosine (Tyr),
(c) Xaa3 is glycine (Gly) or arginine (Arg),
(d) Xaa4 is aspartic acid (Asp), glutamic acid (Glu), or histidine (His),
(e) Xaa5 is serine (Ser), threonine (Thr), or tyrosine (Tyr),
(f) Xaa6 is asparagine (Asn) or glycine (Gly), and
(g) Xaa7 is serine (Ser), alanine (Ala), or aspartic acid (Asp);
or at least CDR1, CDR2, and CDR3 thereof
and the immunoglobulin light chain polypeptide of the IL-36R-binding agent comprises:
(SEQ ID NO: 40)
Asp Ile Val Met Thr Gln Thr Pro Leu Ser Leu Ser
Val Thr Pro Gly Gln Pro Ala Ser Ile Ser Cys Arg
Ser Ser Lys Ser Leu Leu His Xaa1 Asn Xaa2 Ile Thr
Tyr Phe Tyr Trp Tyr Leu Xaa3 Lys Pro Gly Gln Pro
Pro Gln Leu Leu Ile Tyr Gln Met Ser Asn Leu Ala
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Ala Gln Asn
Leu Glu Leu Pro Leu Thr Phe Gly Gly Gly Thr Lys
Val Glu Ile Lys,
(a) Xaa1 is serine (Ser) or arginine (Arg),
(b) Xaa2 is glycine (Gly) or alanine (Ala), and
(c) Xaa3 is glutamine (GM) or histidine (His),
or at least CDR1, CDR2, and CDR3 thereof.

9. The method of claim 7, wherein the immunoglobulin heavy chain polypeptide of the IL-36R-binding agent comprises SEQ ID NO: 22 or at least the CDRs thereof.

10. The method of claim 7, wherein the immunoglobulin light chain polypeptide of the IL-36-binding agent comprises SEQ ID NO: 44 or at least the CDRs thereof.

11. The method of claim 7, wherein the IL-36R-binding agent exhibits one or more of the following biological activities:
(a) inhibits the interaction between IL-36R and IL-36α, IL-36β, and/or IL-36γ,
(b) inhibits intracellular signaling mediated by IL-36R,
(c) cross-reacts with and inhibits the activity of human IL-36R, cynomolgus IL-36R, and non-human primate IL-36R.

12. The method of claim 6, wherein the IL-36R-binding agent is an antibody.

13. The method of claim 6, wherein the IL-36R-binding agent is an antigen-binding antibody fragment.

14. (canceled)

15. (canceled)

16. (canceled)

17. The method of claim 6, wherein the method comprises administering a composition comprising (a) the IL-36R-binding agent, and (b) a pharmaceutically acceptable carrier.

18. (canceled)

19. (canceled)

20. A method of selecting a subject with hidradenitis suppurativa for treatment with an inhibitor of the IL-36 pathway, the method comprising:
(a) comparing the expression of at least one of an IL-36 cytokine, IL-36R, or mRNA encoding same in a tissue or fluid sample from an affected tissue of the subject with a normal control, optionally the level of the same cytokine(s) or receptor or mRNA from the same type of sample from a non-affected tissue of the same subject or from a normal, non-diseased subject, or average of such expression levels in a population of normal, non-diseased subjects); and
(b) selecting the subject for treatment if the expression of the IL-36 cytokine, IL-36R, or mRNA encoding same is increased in the subject as compared to the normal control;
or
(a) comparing the expression of at least one of an IL-36 cytokine, IL-36R, or mRNA encoding same in a tissue or fluid sample from an affected tissue of the subject before and after administration of an IL-36 pathway inhibitor; and
(b) selecting the subject for treatment when a decrease in expression of at least one of an IL-36 cytokine, IL-36R, or mRNA encoding same is observed after administration of the IL-36 pathway inhibitor as compared to before administration of the IL-36 pathway inhibitor.

21. (canceled)

22. The method of claim 20, wherein the IL-36 cytokine is IL-36γ.

23. The method of claim 20, wherein the inhibitor of the IL-36 pathway is an IL-36R-binding agent.

24. The method of claim 20, further comprising treating the hidradenitis in the subject in accordance with the method of claim 1.

25.-26. (canceled)
